SEQUENCE LISTING (12 sequences)

SEQ ID NO: 1 - protein, 442 aa, Homo sapiens
QVQLQQWGAGLLKPSETLSLTCAIYGGSFREYYWSWIRQPPGKGLEWIGEIFHSGSTNYNPSLKSRVTISVDTSKNQFSLKLSSVTAADTAVYYCAREELDDFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 2 - protein, 5 aa, Homo sapiens
EYYWS

SEQ ID NO: 3 - protein, 16 aa, Homo sapiens
EIFHSGSTNYNPSLKS

SEQ ID NO: 4 - protein, 8 aa, Homo sapiens
EELDDFDI

SEQ ID NO: 5 - protein, 116 aa, Homo sapiens
QVQLQQWGAGLLKPSETLSLTCAIYGGSFREYYWSWIRQPPGKGLEWIGEIFHSGSTNYNPSLKSRVTISVDTSKNQFSLKLSSVTAADTAVYYCAREELDDFDIWGQGTMVTVSS

SEQ ID NO: 6 - DNA, 1326 bp, Homo sapiens
  source 1..1326: mol_type = other DNA
caggtgcagctacagcagtggggcgcaggactgttgaagccttcggagaccctgtccctcacctgcgctatctatggtgggtccttcagggagtactactggagctggatccgccagcccccagggaaggggctggagtggattggggaaatctttcatagtggaagcaccaactacaacccgtccctcaagagtcgagtcaccatatcagtagacacgtccaagaaccagttctccctgaagctgagctctgtgaccgccgcggacacggctgtgtattactgtgcgagagaggaattagatgattttgatatctggggccaagggacaatggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtagtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 7 - protein, 214 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYKSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 11 aa, Homo sapiens
RASQGISSWLA

SEQ ID NO: 9 - protein, 7 aa, Homo sapiens
AASSLQS

SEQ ID NO: 10 - protein, 9 aa, Homo sapiens
QQYKSYPRT

SEQ ID NO: 11 - protein, 107 aa, Homo sapiens [sequence omitted]

SEQ ID NO: 12 - DNA, 642 bp, Homo sapiens
  source 1..642: mol_type = other DNA
gacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagtagcctgcagcctgaagattttgcaacttattactgccaacagtataaaagttaccctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

(19) United States
(12) Patent Application Publication
(10) Pub. No.: US 2025/0011417 A1
(43) Pub. Date: Jan. 9, 2025

(54) METHODS FOR THE TREATMENT OF THYROID EYE DISEASE

(71) Applicant: Tourmaline Bio, Inc., New York, NY (US)

(72) Inventors: Sandeep Kulkarni, New York, NY (US); Yung Chyung, New York, NY (US); Susan Dana Jones, New York, NY (US); Ryan Iarrobino, New York, NY (US); W. Bradford Middlekauff, New York, NY (US)

(21) Appl. No.: 18/888,320

(22) Filed: Sep. 18, 2024

Related U.S. Application Data
(63) Continuation of application No. PCT/US2023/079635, filed on Nov. 14, 2023.
(60) Provisional application No. 63/384,878, filed on Nov. 23, 2022.
(60) Provisional application No. 63/493,221, filed on Mar. 30, 2023.

(51) Int. Cl. C07K 16/24; A61K 9/00; A61K 39/00; A61K 47/18; A61K 47/26; A61P 27/02

(52) U.S. Cl. CPC C07K 16/248; A61K 9/0019; A61K 47/183; A61K 47/26; A61P 27/02; A61K 2039/505; A61K 2039/545

(57) ABSTRACT

The present disclosure provides methods of treating thyroid eye disease comprising subcutaneously administering to a patient in need thereof a therapeutically effective dose of an anti-interleukin-6 (anti-IL-6) antibody or antibody fragment. Further provided herein are pharmacologically active agents, compositions, methods and/or dosing schedules for the treatment of thyroid eye disease.

### CROSS-REFERENCE TO RELATED APPLICATIONS

This application claims priority to U.S. Provisional Patent Application No. 63/384,878, filed Nov. 23, 2022, and U.S. Provisional Patent Application No. 63/493,221, filed Mar. 30, 2023, each of which is incorporated by reference herein in its entirety for all purposes.

### DESCRIPTION OF THE TEXT FILE SUBMITTED ELECTRONICALLY

The contents of the electronic sequence listing (TOUR_002_002WO_SeqList_ST26.xml; Size: 14,170 bytes; and Date of Creation: Nov. 9, 2023) are herein incorporated by reference in their entirety.

### TECHNICAL FIELD

The disclosure relates to therapeutic antibody molecules and treatments for thyroid eye disease.

### BACKGROUND

Thyroid eye disease (TED), also called Graves' ophthalmopathy and Graves' orbitopathy, is a disfiguring and potentially sight-threatening autoimmune disease. TED is associated with proliferation and inflammation of the cell types surrounding the eye. Symptoms include proptosis, double-vision, and disfigurement. There are approximately 30,000 new cases per year and about 50% are moderate to severe that warrant treatment with a biologic.

Teprotumumab, an antibody targeting IGF-1 receptor, was approved in January 2020 for the treatment of TED. However, more than 10% of patients receiving teprotumumab have displayed adverse reactions including muscle spasms, alopecia, hearing impairment, and hyperglycemia, which may not always be fully reversible. Moreover, treatment with teprotumumab requires 6 months of intravenous infusions. Roughly half of responders would relapse within 48 weeks of completing the therapy. Thus, there remains a need for development of an effective and well-tolerated treatment with fast onset of activity that is suitable for a longer-term, at-home dosing to reduce the potential for relapse.

### SUMMARY

Provided herein is a method of treating thyroid eye disease (TED) comprising administering to a patient in need thereof a therapeutically effective dose of an anti-interleukin-6 (anti-IL-6) antibody or antibody fragment having the variable heavy (VH) complementarity-determining regions (CDRs) as defined in SEQ ID NOs 2, 3 and 4, and the variable light (VL) CDRs as defined in SEQ ID NOs 8, 9 and 10.

In some embodiments, the anti-IL-6 antibody or antibody fragment comprises a heavy chain polypeptide comprising a polypeptide having at least about 95% identity to SEQ ID NO: 1 and a light chain polypeptide comprising a polypeptide having at least about 95% identity to SEQ ID NO: 7. In one aspect, the anti-IL-6 antibody or antibody fragment comprises a heavy chain polypeptide having the sequence of SEQ ID NO: 1 and a light chain polypeptide having the sequence of SEQ ID NO: 7.

In some embodiments, the anti-IL-6 antibody or antibody fragment containing said CDRs as described herein is contained in a pharmaceutical composition that comprises said anti-IL6 antibody or antibody fragment and a pharmaceutically acceptable carrier. In some embodiments, the pharmaceutical composition comprises 85 mg/mL of the anti-IL-6 antibody, 20 mM histidine, 63.2 mg/mL of sucrose, 16.8 mg/mL of mannitol, 0.05 mg/mL of EDTA, and 0.2 mg/mL of polysorbate 80.

In some embodiments, the therapeutically effective dose of the present disclosure is between 5 mg to 200 mg. In some embodiments, the therapeutically effective dose is about 5, about 7.5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, or about 200 mg of the anti-IL-6 antibody or antibody fragment.

In some embodiments, therapeutically effective dose of the antibody, or an antigen binding fragment thereof may be administered by any suitable route including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, hypospray, intravaginal or rectal routes. In one embodiment, the therapeutically effective dose is administered subcutaneously.

In some embodiments, the dosing schedules for the anti-IL-6 antibody or antibody fragment is every 1 week to every 24 weeks. In one embodiment, the therapeutically effective dose is administered every 4, 8, 12 or 24 weeks.

In some embodiments, the treatment may be provided over a total duration of about 1 month, about 2 months, about 3 months, about 4 months, about 5 months, about 6 months, about 7 months, about 8 months, about 9 months, about 10 months, about 11 months, about 12 months, about 14 months, about 16 months, about 18 months, about 20 months, about 22 months or about 24 months.

In some embodiments, the method of the present disclosure comprises administering the anti-IL-6 antibody at a dose of 50 mg or 20 mg every 8 weeks. In some embodiments, the patient receives 3 doses of treatment.

In some embodiments, the method of the present disclosure comprises: (a) administering a loading dose of the anti-IL-6 antibody or antibody fragment to the patient for at least the first does during a loading regimen; and (b) thereafter administering a maintenance dose of the anti-IL-6 antibody or antibody fragment subcutaneously to the patient during a maintenance regimen. In some embodiments, the loading regimen comprises administering the loading dose every 1 week, every 2 weeks, or every 4 weeks. In some embodiments, the maintenance regimen comprises administering the maintenance dose every 4 weeks, every 8 weeks, every 12 weeks, or every 24 weeks. In some embodiments, the loading dose is greater than or equal to the maintenance dose. In some embodiments, the loading dose is less than the maintenance dose. In some embodiments, the loading dose is between 5 mg to 200 mg. In some embodiments, the maintenance dose is between 5 mg to 200 mg. In one embodiment, the loading regimen comprises one loading dose of 50 mg, and the maintenance regimen comprises the maintenance dose of 20 mg every 4 weeks for a total of 24 weeks. In another embodiment, the loading regimen comprises one loading dose of 20 mg, and the maintenance regimen comprises the maintenance dose of 10 mg every 4 weeks for a total of 24 weeks

In some embodiments, the patient being treated in accordance with methods of the disclosure has Graves' disease associated with active TED. In some embodiments, the patient has an eye with a Clinical Activity Score (CAS) of ≥4 before treatment. In some embodiments, the patient has an eye with proptosis of ≥3 mm above a normal range (based upon race and gender) before treatment. In some embodiments, the patient has thyroid stimulating immunoglobulin (TSI) of >130% of a normal range before treatment. In some embodiments, the patient is euthyroid or has mild hypo- or hyperthyroidism. In some embodiments, the patient has a body mass index of ≤35.0 kg/m².

In some embodiments, the method of treatment as described herein achieves one or more of the following results:
(a) a proptosis reduction of ≥2 mm from baseline in the first eye, without a proptosis increase of ≥2 mm in the second eye;
(b) a Clinical Activity Score (CAS) (7-point scale) of ≤1 in the first eye, without ≥2 point increase in CAS from baseline in the second eye;
(c) a diplopia grade reduction by at least 1 using the Gorman diplopia scale;
(d) improvement from baseline in Graves' ophthalmopathy quality of life (GO-QoL) from baseline by at least 6, 8, 10, 15, or 20 points; or
(e) a reduction in the titter of an autoantibody.

As used herein, the eye with more severe symptoms (based upon CAS and proptosis measurement) is designated as the first eye, and the eye with less severe symptoms is designated as the second eye. The eye with more severe symptoms (referred to as the first eye) is used as a baseline for evaluating the effectiveness of the treatment. In cases where both eyes are equally affected, either one can be designated as the first eye and the other as the second eye.

In some embodiments, the reduction in proptosis could be greater than about 1.5 mm, for example, about 1.8 mm, about 2 mm, about 2.2 mm, about 2.4 mm, about 2.5 mm, about 2.6 mm, about 2.8 mm, about 3 mm, about 3.2 mm, about 3.4 mm, about 3.5 mm, about 3.6 mm, about 3.8 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, or about 5 mm.

In some embodiments, CAS is reduced in the patient to either one (1) or zero (0) (on the 7-point version of the CAS scale). In some embodiments, the reduction in CAS is by 2 points or more, for example, by 3, 4, 5, 6, or 7 points.

In some embodiments, the diplopia grade is reduced by at least 1 using the Gorman diplopia scale. In some embodiments, the reduction in severity of diplopia is sustained at least 20, 30, 40, or 50 weeks after discontinuation of antibody administration. In some embodiments, the reduction in severity of diplopia is sustained 20-30, 30-40, 40-50, or 50-60 weeks after discontinuation of antibody administration. In some embodiments, the reduction in severity of diplopia is sustained at least 20 weeks after discontinuation of antibody administration. In some embodiments, the reduction in severity of diplopia is sustained at least 50 weeks after discontinuation of antibody administration. In some embodiments, the efficacy effect of the treatment is sustained for at least 4 weeks, 8 weeks, 12 weeks, 16 weeks, 24 weeks, 36 weeks, 48 weeks, or 72 weeks after the last dose administration. In some embodiments, the probability of relapse (i.e., loss of proptosis response or CAS response or diplopia response) is less than 40%, 30%, 20%, 15%, 10%, or 5% after one year of the treatment.

In some embodiments, the titter of autoantibodies including thyroid stimulating immunoglobulin (TSI) and/or anti-thyroid stimulating hormone receptor (TSHR) antibodies is reduced by at least 30%, 40%, 50%, 60%, 70%, 80%, or 90%.

In some embodiments, when administered to a population of patients, about 20%, about 25%, about 30%, about 35%, about 40%, about 45%, about 50%, about 55%, about 60%, about 65%, about 70%, about 75%, about 80%, about 85%, about 90%, about 95% or about 100% of the patients may respond with a reduction in proptosis of ≥2 mm from the baseline in the first eye, without a proptosis increase of ≥2 mm in the second eye, and/or a CAS of ≤1 in the first eye, without ≥2 point increase in CAS from baseline in the second eye, and/or a diplopia grade reduction by at least 1. In some embodiments, the probability of the proptosis reduction of ≥2 mm from the baseline in the first eye, without a proptosis increase of ≥2 mm in the second eye, is at least 40%, 50% or 60% or 70%, 75%, or 80%. In some embodiments, the probability of attaining a CAS of ≤1 in the first eye, without ≥2 point increase in CAS from the baseline in the second eye is at least 50%, 60%, 70%, 75% or 80% or 85% or 90%. In some embodiments, the probability of resolution of inconstant diplopia is at least 30%, 40%, 50%, 60% 70%, 80%, or 90%. In some embodiments, the probability of the constant diplopia reduction is at least 30%, 40%, 50%, 60% 70%, 80%, or 90%. In some embodiments, the probability of resolution of constant diplopia to either inconstant diplopia, intermittent diplopia or no diplopia is at least 30%, 40%, 50%, 60% 70%, 80%, or 90%. In some embodiments, the probability of decrease in the number of patients with any diplopia (intermittent, inconstant or constant) is at least 30%, 40%, 50%, 60% 70%, 80%, or 90%. In some embodiments, the probability of decrease in the number of patients with inconstant or constant diplopia is at least 30%, 40%, 50%, 60% 70%, 80%, or 90%. In some embodiments, the probability of decrease in the number of patients with intermittent diplopia is at least 30%, 40%, 50%, 60% 70%, 80%, or 90%.

In some embodiments, the probability of decrease in the number of patients with inconstant diplopia is at least 30%, 40%, 50%, 60% 70%, 80%, or 90%. In some embodiments, the probability of decrease in the number of patients with constant diplopia is at least 30%, 40%, 50%, 60% 70%, 80%, or 90%.

In some embodiments, the treatment result is achieved within 72 weeks, 64 weeks, 56 weeks, 48 weeks, 44 weeks, 40 weeks, 32 weeks, 20 weeks, 16 weeks, 12 weeks, 8 weeks, or 4 weeks. In some embodiments, the treatment result is achieved during a long-term treatment, wherein the long-term is more than 72 weeks.

In some embodiments, the methods described herein further comprise a step of treating a subject with an additional form of therapy. In some embodiments, the additional form of therapy comprises administering one or more therapeutic agent in addition to the anti-IL-6 antibody or antibody fragment as described herein. The therapeutic agents include, but are not limited to, a second antibody (e.g., an anti-IL-1 antibody, anti-IGF-1 receptor antibody, anti-VEGF antibody, and/or anti-IL17a antibody), a soluble receptor (e.g., soluble IL-1 receptor, soluble TNF-alpha receptor), an anti-inflammatory agent (e.g., paclitaxel, docetaxel, cisplatin, doxorubicin, prednisone, mitomycin, progesterone, tamoxifen, or fluorouracil), or a thyroid eye disease medication (e.g., vitamins such as selenium or vitamin D; tropical medicines such as loteprednol or fluorometholone; steroids such as glucocorticoids; or orbital radiation).

In some embodiments, provided are pharmacologically active agents, compositions, methods and/or dosing schedules that have certain advantages compared to the agents, compositions, methods and/or dosing schedules that are currently used and/or known in the art, including the ability to dose less frequently or to administer lower doses to obtain equivalent effects in inhibiting IL-6 mediated signaling.

### BRIEF DESCRIPTION OF THE DRAWINGS

FIG. 1 illustrates the schematic for a multicenter, Phase 2b randomized, double-masked, placebo-controlled, dose-ranging study of TOUR006 with treatment extension in patients with thyroid eye disease. Note: * indicates that this dose may be decreased to 20 mg q8w based upon any emerging safety insights (such as from a periodic safety review by the DSMB) or based upon the results of the primary analysis from Period A.

FIG. 2 presents the alternative schematic for a multicenter, Phase 2b randomized, double-masked, placebo-controlled, dose-ranging study of TOUR006 with treatment extension in patients with thyroid eye disease.

FIG. 3A and FIG. 3B illustrate the predicted percentage changes of CRP over the treatment period for population A (FIG. 3A) and population B (FIG. 3B), respectively, under the dose regimen of 50 mg LD, followed by 20 mg Q4W starting at 4 weeks.

FIG. 4A and FIG. 4B illustrate the predicted percentage changes of CRP over the treatment period for population A (FIG. 4A) and population B (FIG. 4B), respectively, under the dose regimen of 20 mg LD, followed by 10 mg Q4W starting at 4 weeks.

### DETAILED DESCRIPTION

Provided herein are methods of treating thyroid eye disease (TED) comprising subcutaneously administering to a patient in need thereof a therapeutically effective dose of an anti-interleukin-6 (anti-IL-6) antibody or antibody fragment.

Further provided herein are pharmacologically active agents, compositions, methods and/or dosing schedules for the treatment of thyroid eye disease.

### Antibodies

Provided herein are antibodies and antigen-binding fragments thereof that specifically bind IL-6. Antibodies and antigen-binding fragments disclosed herein specifically bind human IL-6. In some embodiments, an antibody may be specific for only human IL-6 and may exhibit no non-human cross-reactivity.

As used herein, the term “antibody” refers to immunoglobulin (Ig) molecules and immunologically active portions or fragments of immunoglobulin molecules, i.e., molecules that contain an antigen-binding site that specifically binds (immunoreacts with) an antigen (e.g., IL-6). By “specifically binds” or “immunoreacts with” is meant that the antibody reacts with one or more antigenic determinants of the desired antigen and does not react with other polypeptides. In some embodiments, an antibody is said to specifically bind an antigen when it preferentially recognizes its target antigen in a complex mixture of proteins and/or macromolecules. In some embodiments, an antibody “specifically binds” IL-6 if the antibody binds IL-6 with greater affinity, greater avidity, more readily and/or for greater duration than it binds other polypeptides.

The term “antibody” broadly refers to an immunoglobulin (Ig) molecule, generally, comprising four polypeptide chains, two heavy (H) chains and two light (L) chains, or any functional fragment, mutant, variant, or derivative thereof, that retains the essential target binding features of an Ig molecule. Such mutant, variant, or derivative antibody formats are known in the art.

In a full-length antibody, each heavy chain comprises a heavy chain variable domain (abbreviated herein as VH domain) and a heavy chain constant region. The heavy chain constant region comprises three domains, CH1, CH2 and CH3. Each light chain comprises a light chain variable domain (abbreviated herein as VL domain) and a light chain constant region. The light chain constant region comprises one domain, CL. The VH and VL domains can be further subdivided into regions of hypervariability, termed complementarity determining regions (CDRs), interspersed with regions that are more conserved, termed framework regions (FRs). Each VH domain and VL domain is composed of three CDRs and four FRs, arranged from amino-terminus to carboxyl-terminus in the following order: FR1, CDR1, FR2, CDR2, FR3, CDR3, FR4.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is according to the EU numbering system. The Fc region of an immunoglobulin generally comprises two constant domains, CH2 and CH3. An Fc region can be present in dimer or monomeric form. The Fc region binds to various cell receptors, such as Fc receptors, and other immune molecules, such as complement proteins.

Immunoglobulin molecules can be of any type (e.g., IgG, IgE, IgM, IgD, IgA or IgY) and class (e.g., IgG1, IgG2, IgG3, IgG4, IgA1 or IgA2) or subclass. IgG, IgD, and IgE antibodies generally contain two identical heavy chains and two identical light chains and two antigen combining domains, each composed of a VH and a VL. Generally IgA antibodies are composed of two monomers, each monomer composed of two heavy chains and two light chains (as for IgG, IgD, and IgE antibodies); in this way the IgA molecule has four antigen binding domains, each again composed of a VH and a VL. Certain IgA antibodies are monomeric in that they are composed of two heavy chains and two light chains. Secreted IgM antibodies are generally composed of five monomers, each monomer composed of two heavy chains and two light chains (as for IgG and IgE antibodies). Thus, the IgM molecule has ten antigen binding domains, each again composed of a VH and a VL. A cell surface form of IgM has a two heavy chain/two light chain structure similar to IgG, IgD and IgE antibodies.

The term “antigen-binding portion” or “antigen-binding fragment” of an antibody (or “antibody portion” or “antibody fragment”), as used herein, refers to one or more fragments of an antibody that retain the ability to specifically bind to an antigen (e.g., IL-6). It has been shown that the antigen-binding function of an antibody can be performed by portions or fragments of a full-length antibody. Examples of binding fragments encompassed within the term “antigen binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb (domain antibody) fragment (Ward et al., (1989) Nature 341:544-546; WO 90/05144 A1, each herein incorporated by reference in its entirety), which comprises a single variable domain; and (vi) an isolated complementarity determining region (CDR). The disclosure also encompasses a Fab′ fragment. Fab′ fragments can be formed by the reduction of F(ab′)₂ fragments. Fab′ derived from F(ab′)₂; therefore, it may contain a small portion of Fc. Furthermore, although the two domains of the Fv fragment, VL and VH, are coded for by separate genes, they can be joined, using recombinant methods, by a synthetic linker that enables them to be made as a single protein chain in which the VL and VH domains pair to form monovalent molecules (known as single chain Fv (scFv). See e.g., Bird et al. (1988) Science 242:423-426; Huston et al. (1988) Proc. Natl. Acad. Sci. USA 85:5879-5883. Such single chain antibodies are also intended to be encompassed within the term “antigen-binding portion” of an antibody. In some embodiments, scFv molecules may be incorporated into a fusion protein. In some embodiments, provided herein is a single chain camelid antibody. In some embodiments, provided herein is a shark heavy chain antibody (V-NAR). See, English et al. (2020) Antibody Therapeutics, 3(1):1-9. Examples of antigen-binding portions are known in the art (Kontermann and Dubel eds., Antibody Engineering (2001) Springer-Verlag. New York. 790 pp.). In some embodiments, provided herein is a single domain antibody. In general, the term “antibody” when used herein encompasses an “antibody fragment”. An antibody fragment generally retains the antigen-binding properties of a full-length antibody.

Antibodies and antibody portions provided herein may be in multispecific (e.g., bispecific or trispecific) formats. Such multispecific molecules specifically bind to two or more different molecular targets or epitopes. In some embodiments, an antibody or an antigen-binding portion is a bispecific molecule that binds specifically to a first antigen and a second antigen, wherein the first antigen is IL-6 and the second antigen is not IL-6. In some embodiments, an antibody or an antigen-binding portion is a diabody. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger et al. (1993) Proc. Natl. Acad. Sci. USA 90:6444-6448; Poljak et al. (1994) Structure 2:1121-1123). In some embodiments, an antibody or an antigen-binding portion is a triabody, a tetrabody, a bis-scFv or a tandem scFv. In some embodiments, an antibody or an antigen-binding portion is a dual affinity re-targeting protein.

In some embodiments, an anti-IL-6 antigen-binding portion disclosed herein is a Fab, a F(ab′)₂, a Fab′, a Fv, a scFv, a Fd, a single domain antibody, a single chain camelid antibody, a diabody, a triabody, a tetrabody or a bis-scFv.

As used herein, the terms “immunological binding” and “immunological binding properties” refer to the non-covalent interactions of the type which occur between an immunoglobulin molecule (e.g., antibody or antigen-binding portion thereof) and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See, Malmqvist, Nature 361:186-187 (1993)). The ratio of K_{off}/Kₒₙ enables the cancellation of all parameters not related to affinity, and is equal to the dissociation constant K_{d}. (See, Davies et al. (1990) Annual Rev Biochem 59:439-473). An antibody or antigen-binding portion provided herein is said to specifically bind IL-6 when the equilibrium binding constant (K_{d}) is ≤10 μM, preferably ≤10 nM, more preferably ≤10 nM, and most preferably ≤100 μM to about 1 μM, as measured by assays such as radioligand binding assays or similar assays known to those skilled in the art.

In some embodiments, an anti-IL-6 antibody or antigen-binding portion provided herein is monovalent or bivalent and comprises a single or double chain. Functionally, the binding affinity of an antibody or antigen-binding portion may be within the range of about 10⁻⁵M to 10⁻¹² M. For example, the binding affinity of an antibody or antigen-binding portion is from about 10⁻⁶ M to 10⁻¹² M, from about 10⁻⁷ M to 10⁻¹² M, from about 10⁻⁸ M to 10⁻¹² M, from about 10⁻⁹ M to 10⁻¹² M, from about 10⁻⁵ M to 10⁻¹¹ M, from about 10⁻⁶ M to 10⁻¹¹ M, from about 10⁻⁷ M to 10⁻¹¹ M, from about 10⁻⁸ M to 10⁻¹¹ M, from about 10⁻⁹ M to 10⁻¹¹ M, from about 10⁻¹⁰ M to 10⁻¹¹ M, from about 10⁻⁵ M to 10⁻¹⁰ M, from about 10⁻⁶ M to 10⁻¹⁰ M, from about 10⁻⁷ M to 10⁻¹⁰ M, from about 10⁻⁸ M to 10⁻¹⁰ M, from about 10⁻⁹ M to 10⁻¹⁰ M, from about 10⁻⁵ M to 10⁻⁹ M, from about 10⁻⁶ M to 10⁻⁹ M, from about 10⁻⁷ M to 10⁻⁹ M, from about 10⁻⁸ M to 10⁻⁹ M, from about 10⁻⁵ M to 10⁻⁸ M, from about 10⁻⁶ M to 10⁻⁸ M, from about 10⁻⁷ M to 10⁻⁸ M, from about 10⁻⁵ M to 10⁻⁷ M, from about 10⁻⁶ M to 10⁻⁷ M or from about 10⁻⁵ M to 10⁻⁶ M.

A human anti-IL-6 monoclonal antibody (PF-04236921) was described in U.S. Pat. No. 8,188,235, the content of which is incorporated herein by reference in its entirety. The human anti-IL-6 monoclonal antibody is a fully human immunoglobulin G2 monoclonal antibody that binds to human IL-6 and has a half-life of 36-51 days. In phase I trials in healthy volunteers and patients with rheumatoid arthritis (protocol B0151001, NCT00838565 and NCT01166555), intravenous and subcutaneous (SC) administration of the human anti-IL-6 monoclonal antibody (PF-04236921) was well tolerated and caused sustained suppression of C-reactive protein (CRP), a marker for inflammation that is transcriptionally controlled by IL-6. PF-04236921 has also been investigated in a phase II trial in patients with systemic lupus erythematosus (SLE; NCT01405196). While the study did not meet the primary end point, improvement was noted in the primary as well as key secondary end points with 10 mg. In 448 subjects who have received treatment with the anti-IL-6 antibody, only 2 subjects have tested positive for the presence of anti-drug antibodies. Overall, the human anti-IL-6 monoclonal antibody demonstrated desirable pharmacokinetic (PK) and pharmacodynamic (PD) properties supporting sustained target inhibition, and low incidence of immunogenicity upon single and multiple dose administration (Danese et al., Gut 2019; 68:40-48; Li et al., Br J Clin Pharmacol. 2018 September; 84(9): 2059-2074.).

TABLE 1
Amino acid and nucleic acid sequences of
human anti-IL-6 antibody

| | Anti-IL-6 antibody (TOUR006) | |
|---|---|---|
| Antibody | | SEQ |
| name | | ID |
| Domain or Region | Sequence | NO |
| Heavy chain | QVQLQQWGAGLLKPSETLSLTCAIY | 1 |
| (amino acid | GGSFREYYWSWIRQPPGKGLEWIGE | |
| sequence) | IFHSGSTNYNPSLKSRVTISVDTSK | |
| | NQFSLKLSSVTAADTAVYYCAREEL | |
| | DDEDIWGQGTMVTVSSASTKGPSVF | |
| | PLAPCSRSTSESTAALGCLVKDYFP | |
| | EPVTVSWNSGALTSGVHTFPAVLQS | |
| | SGLYSLSSVVTVPSSNFGTQTYTCN | |
| | VDHKPSNTKVDKTVERKCCVECPPC | |
| | PAPPVAGPSVFLFPPKPKDTLMISR | |
| | TPEVTCVVVDVSHEDPEVQFNWYVD | |
| | GVEVHNAKTKPREEQFNSTFRVVSV | |
| | LTVVHQDWLNGKEYKCKVSNKGLPA | |
| | PIEKTISKTKGQPREPQVYTLPPSR | |
| | EEMTKNQVSLTCLVKGFYPSDIAV | |
| | EWESNGQPENNYKTTPPMLDSDGSF | |
| | FLYSKLTVDKSRWQQGNVFSCSVMH | |
| | EALHNHYTQKSLSLSPGK | |
| Heavy chain CDR1 | EYYWS | 2 |
| Heavy chain CDR2 | EIFHSGSTNYNPSLKS | 3 |
| Heavy chain CDR3 | EELDDFDI | 4 |
| Heavy chain | QVQLQQWGAGLLKPSETLSLTCAIY | 5 |
| variable region | GGSFREYYWSWIRQPPGKGLEWIGE | |
| (VH) (amino acid | IFHSGSTNYNPSLKSRVTISVDTSK | |
| sequence) | NQFSLKLSSVTAADTAVYYCAREEL | |
| | DDFDIWGQGTMVTVSS | |
| Heavy chain | caggtgcagctacagcagtggggcg | 6 |
| (nucleic acid | caggactgttgaagccttcggagac | |
| sequence) | cctgtccctcacctgcgctatctat | |
| | ggtgggtccttcagggagtactact | |
| | ggagctggatccgccagcccccagg | |
| | gaaggggctggagtggattggggaa | |
| | atctttcatagtggaagcaccaact | |
| | acaacccgtccctcaagagtcgagt | |
| | caccatatcagtagacacgtccaag | |
| | aaccagttctccctgaagctgagct | |
| | ctgtgaccgccgcggacacggctgt | |
| | gtattactgtgcgagagaggaatta | |
| | gatgattttgatatctggggccaag | |
| | ggacaatggtcaccgtctcctcagc | |
| | ctccaccaagggcccatcggtcttc | |
| | cccctggcgccctgctccaggagca | |
| | cctccgagagcacagcggccctggg | |
| | ctgcctggtcaaggactacttcccc | |
| | gaaccggtgacggtgtcgtggaact | |
| | caggcgctctgaccagcggcgtgca | |
| | caccttcccggctgtcctacagtcc | |
| | tcaggactctactccctcagcagcg | |
| | tagtgaccgtgccctccagcaactt | |
| | cggcacccagacctacacctgcaac | |
| | gtagatcacaagcccagcaacacca | |
| | aggtggacaagacagttgagcgcaa | |
| | atgttgtgtcgagtgcccaccgtgc | |
| | ccagcaccacctgtggcaggaccgt | |
| | cagtcttcctcttccccccaaaacc | |
| | caaggacaccctcatgatctcccgg | |
| | acccctgaggtcacgtgcgtggtgg | |
| | tggacgtgagccacg | |
| | aagaccccgaggtccagttcaactg | |
| | gtacgtggacggcgtggaggtgcat | |
| | aatgccaagacaaagccacgggagg | |
| | agcagttcaacagcacgttccgtgt | |
| | ggtcagcgtcctcaccgtcgtgcac | |
| | caggactggctgaacggcaaggagt | |
| | acaagtgcaaggtctccaacaaagg | |
| | cctcccagcccccatcgagaaaacc | |
| | .atctccaaaaccaaagggcagccc | |
| | cgagaaccacaggtgtacaccctgc | |
| | ccccatcccgggaggagatgaccaa | |
| | gaaccaggtcagcctgacctgcctg | |
| | gtcaaaggcttctaccccagcgaca | |
| | tcgccgtggagtgggagagcaatgg | |
| | gcagccggagaacaactacaagacc | |
| | acacctcccatgctggactccgacg | |
| | gctccttcttcctctacagcaagct | |
| | caccgtggacaagagcaggtggcag | |
| | caggggaacgtcttctcatgctccg | |
| | tgatgcatgaggctctgcacaacca | |
| | ctacacgcagaagagcctctccctg | |
| | tctccgggtaaa | |
| Light chain (amino | DIQMTQSPSSLSASVGDRVTITCRA | 7 |
| acid sequence) | SQGISSWLAWYQQKPEKAPKSLIYA | |
| | ASSLQSGVPSRFSGSGSGTDFTLTI | |
| | SSLQPEDFATYYCQQYKSYPRTFGQ | |
| | GTKVEIKRTVAAPSVFIFPPSDEQL | |
| | KSGTASVVCLLNNFYPREAKVQWKV | |
| | DNALQSGNSQESVTEQDSKDSTYSL | |
| | SSTLTLSKADYEKHKVYACEVTHQG | |
| | LSSPVTKSFNRGEC | |
| Light chain CDR1 | RASQGISSWLA | 8 |
| Light chain CDR2 | AASSLQS | 9 |
| Light chain CDR3 | QQYKSYPRT | 10 |
| Light chain | DIQMTQSPSSLSASVGDRVTITCRA | 11 |
| variable | SQGISSWLAWYQQKPEKAPKSLIYA | |
| region (VL) | ASSLQSGVPSRFSGSGSGTDFTLTI | |
| (amino | SSLQPEDFATYYCQQYKSYPRTFGQ | |
| acid sequence) | GTKVEIK | |
| Light chain | gacatccagatgacccagtctccat | 12 |
| (nucleic | cctcactgtctgcatctgtaggaga | |
| acid sequence) | cagagtcaccatcacttgtcgggcg | |
| | agtcagggtattagcagctggttag | |
| | cctggtatcagcagaaaccagagaa | |
| | agcccctaagtccctgatctatgct | |
| | gcatccagtttgcaaagtggggtcc | |
| | catcaaggttcagcggcagtggatc | |
| | tgggacagatttcactctcaccatc | |
| | agtagcctgcagcctgaagattttg | |
| | caacttattactgccaacagtataa | |
| | aagttaccctcggacgttcggccaa | |
| | gggaccaaggtggaaatcaaacgaa | |
| | ctgtggctgcaccatctgtcttcat | |
| | cttcccgccatctgatgagcagttg | |
| | aaatctggaactgcctctgttgtgt | |
| | gcctgctgaataacttctatcccag | |
| | agaggccaaagtacagtggaaggtg | |
| | gataacgccctccaatcgggtaact | |
| | cccaggagagtgtcacagagcagga | |
| | cagcaaggacagcacctacagcctc | |
| | agcagcaccctgacgctgagcaaag | |
| | cagactacgagaaacacaaagtcta | |
| | cgcctgcgaagtcacccatcagggc | |
| | ctgagctcgcccgtcacaaagagct | |
| | tcaacaggggagagtgt | |

CDR1, CDR2 and CDR3 (from left to right) sequences are underlined in the heavy chain and light chain, respectively.

Provided herein is a method of treating thyroid eye disease comprising subcutaneously administering to a patient in need thereof a therapeutically effective dose of an anti-interleukin-6 (anti-IL-6) antibody or antibody fragment having the variable heavy (VH) CDRs as defined in SEQ ID NOs 2, 3 and 4, and the variable light (VL) CDRs as defined in SEQ ID NOs 8, 9 and 10. In some embodiments, said antibody or antibody fragment comprises a heavy chain polypeptide comprising a polypeptide having at least about 95%, about 96%, about 97%, about 98% or about 99% identity to SEQ ID NO: 1 and a light chain polypeptide comprising a polypeptide having at least about 95%, about 96%, about 97%, about 98% or about 99% identity to SEQ ID NO: 7. In some embodiments, said antibody or antibody fragment comprises a heavy chain polypeptide comprising a polypeptide having the sequence of SEQ ID NO: 1 and a light chain polypeptide comprising a polypeptide having the sequence of SEQ ID NO: 7. In some embodiments, the anti-IL-6 antibody or an antigen-binding portion comprises human IgG2 constant regions.

As used herein, the term “conservative substitution” refers to replacement of an amino acid with another amino acid which does not significantly deleteriously change the functional activity. A preferred example of a “conservative substitution” is the replacement of one amino acid with another amino acid which has a value ≥0 in the following BLOSUM 62 substitution matrix (see Henikoff & Henikoff, 1992, PNAS 89: 10915-10919):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | −1 | −2 | −2 | 0 | −1 | −1 | 0 | −2 | −1 | −1 | −1 | −1 | −2 | −1 | 1 | 0 | −3 | −2 | 0 |
| R | −1 | 5 | 0 | −2 | −3 | 1 | 0 | −2 | 0 | −3 | −2 | 2 | −1 | −3 | −2 | −1 | −1 | −3 | −2 | −3 |
| N | −2 | 0 | 6 | 1 | −3 | 0 | 0 | 0 | 1 | −3 | −3 | 0 | −2 | −3 | −2 | 1 | 0 | −4 | −2 | −3 |
| D | −2 | −2 | 1 | 6 | −3 | 0 | 2 | −1 | −1 | −3 | −4 | −1 | −3 | −3 | −1 | 0 | −1 | −4 | −3 | −3 |
| C | 0 | −3 | −3 | −3 | 9 | −3 | −4 | −3 | −3 | −1 | −1 | −3 | −1 | −2 | −3 | −1 | −1 | −2 | −2 | −1 |
| Q | −1 | 1 | 0 | 0 | −3 | 5 | 2 | −2 | 0 | −3 | −2 | 1 | 0 | −3 | −1 | 0 | −1 | −2 | −1 | −2 |
| E | −1 | 0 | 0 | 2 | −4 | 2 | 5 | −2 | 0 | −3 | −3 | 1 | −2 | −3 | −1 | 0 | −1 | −3 | −2 | −2 |
| G | 0 | −2 | 0 | −1 | −3 | −2 | −2 | 6 | −2 | −4 | −4 | −2 | −3 | −3 | −2 | 0 | −2 | −2 | −3 | −3 |
| H | −2 | 0 | 1 | −1 | −3 | 0 | 0 | −2 | 8 | −3 | −3 | −1 | −2 | −1 | −2 | −1 | −2 | −2 | 2 | −3 |
| I | −1 | −3 | −3 | −3 | −1 | −3 | −3 | −4 | −3 | 4 | 2 | −3 | 1 | 0 | −3 | −2 | −1 | −3 | −1 | 3 |
| L | −1 | −2 | −3 | −4 | −1 | −2 | −3 | −4 | −3 | 2 | 4 | −2 | 2 | 0 | −3 | −2 | −1 | −2 | −1 | 1 |
| K | −1 | 2 | 0 | −1 | −3 | 1 | 1 | −2 | −1 | −3 | −2 | 5 | −1 | −3 | −1 | 0 | −1 | −3 | −2 | −2 |
| M | −1 | −1 | −2 | −3 | −1 | 0 | −2 | −3 | −2 | 1 | 2 | −1 | 5 | 0 | −2 | −1 | −1 | −1 | −1 | 1 |
| F | −2 | −3 | −3 | −3 | −2 | −3 | −3 | −3 | −1 | 0 | 0 | −3 | 0 | 6 | −4 | −2 | −2 | 1 | 3 | −1 |
| P | −1 | −2 | −2 | −1 | −3 | −1 | −1 | −2 | −2 | −3 | −3 | −1 | −2 | −4 | 7 | −1 | −1 | −4 | −3 | −2 |
| S | 1 | −1 | 1 | 0 | −1 | 0 | 0 | 0 | −1 | −2 | −2 | 0 | −1 | −2 | −1 | 4 | 1 | −3 | −2 | −2 |
| T | 0 | −1 | 0 | −1 | −1 | −1 | −1 | −2 | −2 | −1 | −1 | −1 | −1 | −2 | −1 | 1 | 5 | −2 | −2 | 0 |
| W | −3 | −3 | −4 | −4 | −2 | −2 | −3 | −2 | −2 | −3 | −2 | −3 | −1 | 1 | −4 | −3 | −2 | 11 | 2 | −3 |
| Y | −2 | −2 | −2 | −3 | −2 | −1 | −2 | −3 | 2 | −1 | −1 | −2 | −1 | 3 | −3 | −2 | −2 | 2 | 7 | −1 |
| V | 0 | −3 | −3 | −3 | −1 | −2 | −2 | −3 | −3 | 3 | 1 | −2 | 1 | −1 | −2 | −2 | 0 | −3 | −1 | 4 |

Calculations of sequence homology or identity (the terms are used interchangeably herein) between sequences may be performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least about 30%, preferably at least about 40%, more preferably at least about 50%, even more preferably at least about 60%, and even more preferably at least about 70%, about 75%, about 80%, about 82%, about 84%, about 85%, about 86%, about 87%, about 88%, about 89%, about 90%, about 91%, about 92%, about 93%, about 94%, about 95%, about 96%, about 97%, about 98%, about 99%, or about 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, considering the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman et al. ((1970) J. Mol. Biol. 48:444-453) algorithm which has been incorporated into the GAP program in the GCG software package, using either a BLOSUM 62 matrix or a PAM250 matrix, and a gap weight of 16, 14, 12, 10, 8, 6, or 4 and a length weight of 1, 2, 3, 4, 5, or 6. In some embodiments, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. One set of parameters (and the one that can be used if the practitioner is uncertain about what parameters should be applied to determine if a molecule is within a sequence identity or homology limitation of the invention) is a BLOSUM 62 scoring matrix with a gap penalty of 12, a gap extend penalty of 4, and a frameshift gap penalty of 5.

In some embodiments, the percent identity between two amino acid or nucleotide sequences can be determined using the algorithm of Meyers et al. ((1989) CABIOS 4:11-17) which has been incorporated into the ALIGN program (version 2.0), using a PAM120 weight residue table, a gap length penalty of 12 and a gap penalty of 4.

In some embodiments, the anti-IL-6 antibody or antigen-binding portion provided herein is monoclonal.

In some embodiments, the anti-IL-6 antibody or antigen-binding portion provided herein is chimeric. The term “chimeric” is intended to refer to an antibody molecule, or an antigen-binding portion thereof, in which the variable domain sequences are derived from one species and at least one constant region sequence is derived from another species. For example, one or all the variable domains of the light chain(s) and/or one or all the variable domains of the heavy chain(s) of a mouse antibody (e.g., a mouse monoclonal antibody) may each be joined to a human constant region, such as, without limitation an IgG1, IgG2, or IgG4 human constant region. Examples of chimeric antibodies and suitable techniques for their generation are provided in U.S. Pat. Nos. 4,816,567; 4,975,369; and 4,816,397, each of which is incorporated herein by reference in its entirety.

In some embodiments, the anti-IL-6 antibody or antigen-binding portion provided herein is humanized. The term “humanized” is intended to refer to an antibody, or an antigen-binding portion thereof, that has been engineered to comprise one or more human framework regions in the variable domain together with non-human (e.g., mouse, rat, or hamster) CDRs of the heavy and/or light chain. In some embodiments, a humanized antibody comprises sequences that are entirely human except for the CDRs. In some embodiments, the VH domain, the VL domain, or both the VH domain and the VL domain of an anti-IL-6 antibody or antigen-binding portion provided herein comprise one or more human framework region amino acid sequences. In some embodiments, a humanized antibody comprises sequences that are entirely human except for the CDRs. Examples of humanized antibodies and suitable techniques for their generation are provided in Hwang et al., Methods 36:35, 2005; Queen et al., Proc. Natl. Acad. Sci. USA, 86:10029-10033, 1989; Jones et al., Nature, 321:522-25, 1986; Riechmann et al., Nature, 332:323-27, 1988; Verhoeyen et al., Science, 239:1534-36, 1988; Orlandi et al., Proc. Natl. Acad. Sci. USA, 86:3833-37, 1989; U.S. Pat. Nos. 5,225,539; 5,530,101; 5,585,089; 5,693,761; 5,693,762; 6,180,370; and WO 90/07861, each of which is incorporated herein by reference in its entirety.

In some embodiments, humanization comprises removal of post-translational modification (PTM) sites in the variable domain sequences (e.g., in the CDR or framework sequences) of a non-human antibody. For example, one or more PTM sites in CDR sequences may be removed by substituting certain amino acid residues. In some embodiments, humanization comprises CDR grafting and back mutation.

In some embodiments, the anti-IL-6 antibody or antigen-binding portion thereof comprises an immunoglobulin constant region. In some embodiments, the immunoglobulin constant region is IgG, IgE, IgM, IgD, IgA or IgY. In some embodiments, the immunoglobulin constant region is IgG1, IgG2, IgG3, IgG4, IgA1 or IgA2. In some embodiments, the immunoglobulin constant region is immunologically inert. In some embodiments, the immunoglobulin constant region comprises one or more mutations to reduce or prevent FcγR binding, antibody-dependent cell-mediated cytotoxicity activity, and/or complement-dependent cytotoxicity activity. In some embodiments, the immunoglobulin constant region is a wild-type human IgG1 constant region, a wild-type human IgG2 constant region, a wild-type human IgG4 constant region, a human IgG1 constant region comprising the amino acid substitutions L234A, L235A and G237A, a human IgG1 constant region comprising the amino acid substitutions L234A, L235A, G237A and P331S or a human IgG4 constant region comprising the amino acid substitution S228P, wherein numbering is according to the EU numbering system. In some embodiments, a position of an amino acid residue in a constant region of an immunoglobulin molecule is numbered according to EU nomenclature (Ward et al., 1995 Therap. Immunol. 2:77-94).

In some embodiments, the anti-IL-6 antibody or antigen-binding portion thereof may comprise an immunoglobulin light chain constant region that is a kappa light chain constant region or a lambda light chain constant region.

In some embodiments, the anti-IL-6 antibody or antigen-binding portion thereof may comprise a human IgG4 constant region comprising the amino acid substitution S228P and a kappa light chain constant region.

Further provided herein is an immunoconjugate comprising an anti-IL-6 antibody or an antigen-binding portion linked to a therapeutic agent. In some embodiments, the therapeutic agent is a small molecule drug.

### Pharmaceutical Compositions

The anti-IL-6 antibodies and antigen-binding portions described herein (also referred to herein as “active compounds”) can be incorporated into pharmaceutical compositions suitable for administration. TOUR006 may potentially be treated through home administration (either self-administered or by caregiver or by visiting healthcare professional). Such compositions typically comprise an anti-IL-6 antibody or antigen-binding portion (or an immunoconjugate comprising said antibody or portion), and a pharmaceutically acceptable carrier, diluent or excipient. As used herein, the term “pharmaceutically acceptable” refers to molecular entities and compositions that do not generally produce allergic or other serious adverse reactions when administered using routes well known in the art. Molecular entities and compositions approved by a regulatory agency of the U.S. federal or state government or listed in the U.S. Pharmacopeia or other generally recognized pharmacopeia for use in animals, and more particularly in humans are considered to be “pharmaceutically acceptable.” As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Some examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Provided herein is a pharmaceutical composition comprising (i) an anti-IL-6 antibody or an antigen-binding portion thereof, wherein the antibody or antigen-binding portion comprises a VH domain and a VL domain, wherein: (a) the VH domain amino acid sequence comprises HCDR1 of SEQ ID NO: 2, HCDR2 of SEQ ID NO: 3 and HCDR3 of SEQ ID NO: 4; and the VL domain amino acid sequence comprises LCDR1 of SEQ ID NO: 8, LCDR2 of SEQ ID NO: 9 and LCDR3 of SEQ ID NO: 10; and (ii) a pharmaceutically acceptable carrier, diluent or excipient.

A pharmaceutical composition disclosed herein may be formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primojel®, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the compounds may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical agents can also be prepared in the form of suppositories (e.g., with conventional suppository bases such as cocoa butter and other glycerides) or retention enemas for rectal delivery.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

In some embodiments, TOUR006 is formulated at a concentration of 85 mg/mL with 20 mM histidine, 63.2 mg/mL sucrose, 16.8 mg/mL mannitol, 0.05 mg/mL EDTA, and 0.2 mg/mL polysorbate 80, pH 5.8. After reconstitution with water for injection, each single use vial contains 106 mg of TOUR006 in 1.25 mL of aqueous solution.

The pharmaceutical compositions provided herein can be included in a container, pack, or dispenser together with instructions for administration.

### Uses of Antibodies

Provided herein are methods and uses of the anti-IL-6 antibodies, anti-IL-6 antigen-binding portions, immunoconjugates and pharmaceutical compositions described herein for providing a therapeutic benefit to a subject with a condition associated with IL-6 expression. In some embodiments, the condition is thyroid eye disease.

In some embodiments, the methods described herein further comprise a step of treating a subject with an additional form of therapy. In some embodiments, the additional form of therapy comprises administering one or more therapeutic agent in addition to the said anti-IL-6 antibody or antibody fragment as described herein. The therapeutic agents include, but are not limited to, a second antibody (e.g., an anti-IL-1 antibody, anti-IGF-1 receptor antibody, anti-VEGF antibody, and/or anti-IL17a antibody), a soluble receptor (e.g., soluble IL-1 receptor, soluble TNF-alpha receptor), an anti-inflammatory agent (e.g., paclitaxel, docetaxel, cisplatin, doxorubicin, prednisone, mitomycin, progesterone, tamoxifen, or fluorouracil), or a thyroid eye disease medication (e.g., vitamins such as selenium or vitamin D; tropical medicines such as loteprednol or fluorometholone; steroids such as glucocorticoids; or orbital radiation).

Provided herein is an anti-IL-6 antibody or an anti-IL-6 antigen-binding portion, an immunoconjugate or a pharmaceutical composition described herein, for use as a medicament.

As used herein, the term “effective amount” or “therapeutically effective amount” refers to the amount of a pharmaceutical agent, e.g., an anti-IL-6 antibody or an antigen-binding portion thereof, which is sufficient to reduce or ameliorate the severity and/or duration of a disorder, e.g., thyroid eye disease, or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent). In some embodiments, the therapeutically effective dose of said anti-IL-6 antibody or antibody fragment is effective to change one or more biomarkers of IL-6 mediated signaling including, but not limited to, total sIL-6R, total IL-6, C-reactive protein (CRP), an/or autoantibodies, for unexpectedly prolonged periods of time.

As used herein, the terms “treat,” “treating,” “treatment,” and the like refer to reducing or ameliorating a disorder, and/or signs or symptoms associated therewith, or slowing or halting the progression thereof. It will be appreciated that, although not precluded, treating a disorder or condition does not require that the disorder, condition or symptoms associated therewith be completely eliminated.

As used herein, “pre-treatment” means prior to the first administration of an anti-IL-6 antibody according to the methods described herein. Pre-treatment does not exclude, and often includes, the prior administration of treatments other than an anti-IL-6 antibody.

As used herein, “post-treatment” means after the administration of an anti-IL-6 antibody according to the methods described herein. Post-treatment includes after any administration of an anti-IL-6 antibody at any dosage described herein. Post-treatment also includes after the treatment phase of an anti-IL-6 antibody.

The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g. decisions on dosage etc., is within the responsibility of general practitioners and other medical doctors and may depend on the severity of the symptoms and/or progression of a disease being treated. Appropriate doses of antibody molecules are well known in the art (Ledermann et al., 1991, Int. J. Cancer 47: 659-664; Bagshawe et al., 1991, Antibody, Immunoconjugates and Radiopharmaceuticals 4: 915-922). Specific dosages may be indicated herein or in the Physician's Desk Reference (2003) as appropriate for the type of medicament being administered may be used. A therapeutically effective amount or suitable dose of an antibody molecule may be determined by comparing its in vitro activity and in vivo activity in an animal model. Methods for extrapolation of effective dosages in mice and other test animals to humans are known. The precise dose will depend upon a number of factors, including whether the antibody is for prevention or for treatment, the size and location of the area to be treated, the precise nature of the antibody (e.g., whole antibody, fragment) and the nature of any detectable label or other molecule attached to the antibody.

A typical antibody dose will be in the range 100 μg to 1 g for systemic applications, and 1 μg to 1 mg for intradermal injection. In one embodiment, an initial higher loading dose, followed by one or more lower doses, may be administered. In another embodiment, an initial lower loading dose, followed by one or more higher doses, may be administered. In some embodiments, the antibody is a whole antibody, e.g., the IgG1, IgG2 or IgG4 isotype. This is a dose for a single treatment of an adult subject, which may be proportionally adjusted for children and infants, and also adjusted for other antibody formats in proportion to molecular weight. Treatments may be repeated at daily, twice-weekly, weekly or monthly intervals, at the discretion of the physician. The treatment schedule for a subject may be dependent on the pharmacokinetic and pharmacodynamic properties of the antibody composition, the route of administration and the nature of the condition being treated. In some embodiments, the dosing of the present disclosure comprises an amount of at least about 10 mg, or at least about 20 mg, or at least about 30 mg, or at least about 40 mg, or at least about 50 mg of the anti-IL-6 antibody or antibody fragment.

Treatment may be periodic, and the period between administrations may be about two weeks or more, e.g., about three weeks or more, about four weeks or more, about once a month or more, about five weeks or more, or about six weeks or more. For example, treatment may be every two to four weeks or every four to eight weeks. Treatment may be given before, and/or after surgery, and/or may be administered or applied directly at the anatomical site of surgical treatment or invasive procedure. Suitable formulations and routes of administration are described above. In some embodiments, the dosing schedules for the anti-IL-6 antibody or antibody fragment is once every 4 or 8 weeks up to about 52 total weeks.

In some embodiments, a subject is a human, a non-human primate, a pig, a horse, a cow, a dog, a cat, a guinea pig, a mouse or a rat. In some embodiments, a subject is an adult human. In some embodiments, a subject is a pediatric human.

### Thyroid Eye Disease

As used herein, “Thyroid Eye Disease (TED),” “Thyroid-associated Ophthalmopathy (TAO),” “Thyroid Inflammatory Eye Disease (TIED),” “Graves' Ophthalmopathy (GO)” or “Graves' Orbitopathy (GO)” refer to the same disorder or condition and are used interchangeably. They all refer to the inflammatory orbital pathology associated with some autoimmune thyroid disorders, most commonly with “Graves' Disease (GD)”, but sometimes with other diseases, e.g. Hashimoto's thyroiditis.

TED has 2 phases. The first is called the “acute” or “active” phase of TED. The second is called the “chronic” or “inactive” phase of TED. TED is characterized by an active disease phase in which progressive inflammation, swelling, and tissue changes occur. This phase is associated with a variety of symptoms including pain, a gritty feeling in the eyes, swelling or abnormal positioning of the eyelids, watery eyes, bulging eyes (proptosis) and double vision (diplopia). The active phase can last anywhere from approximately 6 months to 3 years. This is followed by an inactive phase in which the disease progression has stopped. However, some symptoms such as double vision and bulging eyes can remain.

Severity of TED can be classified as: (1) Sight-threatening thyroid eye disease; (2) Moderate-to-severe thyroid eye disease; and (3) Mild thyroid eye disease.

Patients with sight-threatening thyroid eye disease have dysthyroid optic neuropathy (DON) and/or corneal breakdown. This category warranted immediate intervention.

Patients with moderate-to-severe thyroid eye disease usually have any one or more of the following: lid retraction ≥2 mm, moderate or severe soft tissue involvement, exophthalmos ≥3 mm above normal for race and gender, inconstant or constant diplopia. Patients' eye conditions have sufficient impact on daily life to justify the risks of immunosuppression (if active) or surgical intervention (if inactive).

Patients with mild thyroid eye disease usually have only one or more of the following: minor lid retraction (<2 mm), mild soft tissue involvement, exophthalmos <3 mm above normal for race and gender, transient or no diplopia, and corneal exposure responsive to lubricants. Patients' eye conditions have only a minor impact on daily life insufficient to justify immunosuppressive or surgical treatment.

As used herein, the terms “proptosis” and “exophthalmos” are used interchangeably and refer to the forward projection, displacement, bulging, or protrusion of the eye anteriorly out of the orbit. Owing to the rigid bony structure of the orbit with only anterior opening for expansion, any increase in orbital soft tissue contents taking place from the side or from behind will displace the eyeball forward. Proptosis or exophthalmos can be the result of a several disease processes including infections, inflammations, tumors, trauma, metastases, endocrine lesions, vascular diseases and extra orbital lesions. The normal range of proptosis is 12-24 mm and the value for a normal person varies by age, gender and race. One of skill in the art, for example an ophthalmologist, surgeon or other clinician skilled in the knowledge and treatment of eye disorders would know what a normal value of proptosis is based on the age, gender and race of the subject and have the ability to diagnose or evaluate the presence or absence of proptosis as well as track its progression. It is generally accepted in the field that a difference greater than 2 mm between the eyes is significant and not normal.

TED is currently recognized as the most common cause of proptosis in adults. Proptosis can be either bilateral, as is often seen in TED, or unilateral (as is often seen in an orbital tumor).

The degree of proptosis can be measured using an exophthalmometer, an instrument used for measuring the degree of forward displacement of the eye. The measurement is the distance between the point on the temporal orbital rim at the deepest palpable point of the angle and the apex of the cornea. Right and left eye readings are performed sequentially without removing the instrument from the orbital rims.

Computed tomography (CT) scanning and Magnetic resonance imaging (MRI) may also be used in evaluating the degree of proptosis. Orbital CT scans are obtained using contiguous axial slices, with the patient's head positioned parallel to the Frankfurt plane. Measurement of proptosis is performed on the CT image by drawing a horizontal line between the lateral orbital rims on an axial plane that bisects the lens and then drawing a perpendicular line forward to the posterior surface of the cornea. MRI may also be used in evaluation due to its multiplanar and inherent contrast capabilities. MRI allows for better soft tissue differentiation in the periorbital, orbital, and intracranial spaces. Use of MRI prevents ionizing radiation of the orbits and risk of radiation-induced cataracts. The imaging findings are similar to those described above for CT.

Orbital ultrasonography can also be used for the diagnosis and evaluation of proptosis. High reflectivity and enlargement of the extraocular muscles are assessed easily by orbital ultrasonography, and serial ultrasonographic examinations can also be used to assess progression or stability of the ophthalmopathy.

Based on the technologies currently available, or that will become available in the future, one of skill in the art would be capable of determining the best modality for diagnosing and evaluating the extent of proptosis or exophthalmos.

The degree of active phase of TED can be assessed by Clinical Activity Score (CAS). This score, based on the classical signs of acute inflammation (pain, redness, swelling, and impaired function) was proposed as a clinical classification to discriminate easily between active and inactive disease (Mourits et al., British Journal of Ophthalmology, 1989, 73, no. 8, 639-644; Mourits et al., Clinical Endocrinology, 1997. 47, no. 1, 9-14).

The 7-point scale CAS consists of seven components: spontaneous retrobulbar pain, pain on attempted eye movements (upward, side-to-side, and downward gazes), conjunctival redness, redness of the eyelids, chemosis, swelling of the caruncle/plica, and swelling of the eyelids. Each component is scored as present or absent, 1 or 0. The score at each efficacy assessment is the sum of all items present. This gives a range of 0-7, where 0 or 1 constitutes inactive disease and 7 severe active ophthalmopathy. A change of >2 points is considered clinically meaningful.

The 10-point scale CAS includes 3 additional components: increase in measured proptosis >2 mm over 1-3 months; decrease in eye movement limit of >8° over 1-3 months; and decrease in visual acuity (2 Snellen chart lines) over 1-3 months.

In addition to proptosis (or exophthalmos) and CAS, quality of life (QoL) may be evaluated with the use of the Graves' ophthalmopathy quality of life (GO-QoL) questionnaire. This questionnaire is designed to determine the improved quality of life after treatment. In some embodiments, questionnaire may determine the decreased or lack of side effects after being treated with an antibody, or an antigen binding fragment thereof, according to the methods disclosed herein, as compared to treatment with glucocorticoids.

The questionnaire has two self-assessment subscales. The first relates to the impact of visual function on daily activities, while the second relates to the impact of self-perceived appearance. Each subscale has 8 questions which are answered with: (i) yes—very much so; (ii) yes—a little; or (iii) no—not at all. Each question is scored 0-2, respectively, and the total raw score is then mathematically transformed to a 0-100 scale, where 0 represents the most negative impact on quality of life, and 100 represents no impact. A change of >8 points on the 0-100 scale is considered to be clinically meaningful. The combined score takes raw scores from both subscales and again transforms them to a single 0-100 scale.

Other grading systems for the assessment of TED include VISA classification (vision, inflammation, strabismus, and appearance) (Dolman and Rootman, Ophthalmic Plastic and Reconstructive Surgery, 2006, 22, no. 5, 319-324 and Dolman, Best Practice & Research Clinical Endocrinology & Metabolism, 2012, 26, no. 3, 229-248), the European Group of Graves' Orbitopathy (EUGOGO) Classification (Bartalena, et al., European Journal of Endocrinology, 2008, 158, no. 3, 273-285), NO SPECS (No physical signs or symptoms, Only signs, Soft tissue involvement, Proptosis, Extraocular muscle signs, Corneal involvement, and Sight loss) Classification system (Dickinson, In: Wiersinga W M, Kahaly G J, eds. Graves' orbitopathy: A multidisciplinary approach—questions and answers, Basel: Karger; 2010:1-25) and Total Motility Score (TMS) (Haggerty et al., Arch Ophthalmol, 2005, 123:356-362).

The VISA system was developed by Dolman and Rootman in 2006 and adopted with modifications by the International Thyroid Eye Disease Society (ITEDS). The VISA system is based on symptoms and signs inputs. The system assesses 4 severity parameters: V (vision); I (inflammation/congestion); S (strabismus/motility restriction); and A (appearance/exposure). Each feature is considered and graded independently. A global severity grade (maximum score is 20 points) is the sum of each of the involved systems graded independently: vision: 1 point; inflammation/congestion: 10 points; strabismus: 6 points (diplopia: 3 points plus restriction: 3 points); appearance/exposure: 3 points.

Vision (V) evaluates the visual repercussion particularly due to the development of dysthyroid optic neuropathy. This is assessed through visual acuity, pupillary reflexes, color vision, visual fields, optic nerve examination, and visual evoked potentials.

The presence of strabismus/motility restriction (S) is documented by three aspects. (1) Changes in diplopia that is assessed using the Gorman subjective diplopia score (range from 0 to 3; 0=no diplopia, 1=diplopia with horizontal or vertical gaze, 2=intermittent diplopia in straight gaze, and 3=constant diplopia in straight gaze). An improvement of ≥1 grade is considered clinically significant. (2) Ocular ductions are measured to the nearest 5° in four directions using the corneal light reflex technique. Accurate assessment of changes in ocular ductions in GO is vital to identify progressive disease, management, and response to therapy assessment. Any change of ≥12° in any direction can be considered progression. (3) Ocular restriction can be graded from 0 to 3 based on the range of ductions (0=duction >45°, 1=30-45°, 2=15-30°, and 3<15°). Strabismus can be quantified by prism cover testing in order to plan surgical treatment.

The European Group on Graves' Orbitopathy (EUGOGO) has stratified disease severity into three categories to help guide treatment. According to the EUGOGO protocol, patients having sight-threatening TED as a result of compressive optic neuropathy or exposure keratopathy may need immediate treatment in the form of systemic corticosteroids or surgery. However, long-term systemic corticosteroids may result in substantial side effects by causing hyperglycemia, hypertension, steroid-induced psychosis, significant weight gain by fluid retention, decreased bone density, gastric discomfort, insomnia and cushingoid features. Further, intravenous corticosteroids may also cause hepatic toxicity, including liver failure. Patients having moderate to severe TED may benefit from immunosuppressive agents or orbital radiotherapy.

Total Motility Score (TMS) is calculated based on the values of ocular ductions, measured (in degrees) in the four main orthogonal gaze directions (up gaze, abduction, downgaze, adduction) with a Forster perimeter arc. (Campi, Thyroid, 2021 February; 31(2): 280-287).

TED is commonly considered to be the autoimmune orbital manifestation of Graves' Disease (GD). GD or thyroid endocrinopathy is characterized by an autoimmune activation of the thyrotropin receptor. It is accepted that the generation of autoantibodies that act as agonists on the thyroid-stimulating hormone receptor (TSHR) is responsible for Graves' hyperthyroidism. Pathogenic overstimulation of TSHR leads to overproduction of thyroid hormones (T3 and T4) and accelerated metabolism of many tissues.

In active TED, autoantibodies trigger connective tissue and fat to expand, in part from stimulating excessive synthesis of hyaluronan. The expanded tissues are infiltrated with T and B cells, become inflamed, and get and extensively remodeled. It has been suggested that TSHR might have some pathogenic role in the development of active TED.

Autoantibodies that bind and transactivate the TSHR lead to stimulation of the thyroid gland independent of the normal feedback-regulated thyroid-stimulating hormone (TSH) stimulation. These TSHR autoantibodies also are known as long-acting-thyroid-stimulator or thyroid-stimulating immunoglobulins (TSI). Some patients with GD also have TSHR-blocking antibodies, which do not transactivate the TSHR. The balance between TSI and TSHR-blocking antibodies, as well as their individual titers, are felt to be determinants of GD severity.

The presence or level of autoantibodies including TSI and TSHR antibodies can be quantified using methods well known in the art, such as Roche Elecsys anti-TSHR assay (ROC-TBII), Quidel Thyretain™ TSI Reporter BioAssay Kit (QUI-TSI), and Otsuka aequorin TSAb assay (OTS-TSI). (Stan et al., Thyroid. 2022 February; 32 (2): 170-176).

As used herein, the term “C-reactive protein (CRP)” refers to a marker of inflammation. CRP levels increase in response to inflammation, and can be measured with a hsCRP (high-sensitivity C-reactive protein) test. The pre-treatment hsCRP of the patients is typically greater than 2 mg/L. Under certain circumstances, the pre-treatment hsCRP level of the patient is 1 mg/L or less.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously.

The disclosure will be further clarified by the following examples, which are intended to be purely exemplary of the disclosure and in no way limiting.

### EXAMPLES

### Example 1: Clinical Evaluation of Human Anti-IL6 Antibody in Patients with Thyroid Eye Disease

This is a multicenter, Phase 2b randomized, double-masked, placebo-controlled, parallel arm study to compare the efficacy and safety of 2 different doses of subcutaneously administered TOUR006 versus placebo (PBO) in the first-line treatment of patients with thyroid eye disease (TED).

### Study Objective

The primary objective of the study is to evaluate the efficacy of TOUR006 in reducing proptosis.

Secondary objectives include the following:
(1) To evaluate the efficacy of TOUR006 in reducing clinical activity score (CAS);
(2) To explore the efficacy of two different dose levels of TOUR006;
(3) To characterize the efficacy of TOUR006 on other outcome measures (3) To characterize the safety of TOUR006; and
(4) To evaluate the long-term efficacy and safety outcomes of TOUR006.

The pharmacokinetic (PK)/pharmacodynamic (PD) objective of the study is to characterize the PK and PD profile and immunogenicity of subcutaneous (SC) delivery of TOUR006.

### Study Design

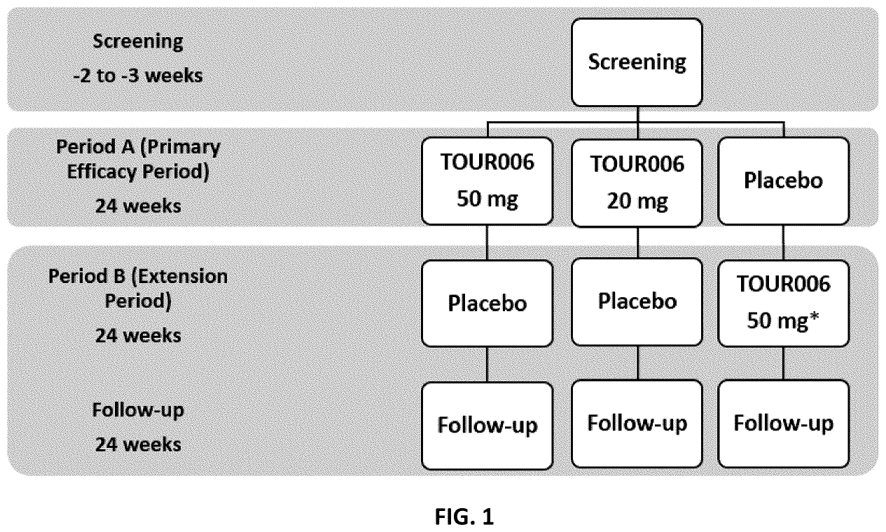

The study schematic is shown in FIG. 1. The total duration of study participation for each participant will be up to approximately 75 weeks. The study will consist of a screening period of up to 3 weeks, followed by a 24-week treatment period (Period A [Primary Efficacy Period]) and a 48-week treatment and follow-up period (Period B [Extension Period]). In Period A, participants will be randomly assigned in a 1:1:1 ratio to receive subcutaneous treatment with TOUR006 20 mg q8w, TOUR006 50 mg q8w, or placebo q8w. Randomization will be stratified by a participant's baseline proptosis (<23 mm versus ≥23 mm). Participants will receive double-masked study intervention during the 3 dosing visits (Day 1, Week 8, and Week 16) with the primary efficacy assessment conducted at Week 20.

Participants will then undergo further study intervention administration and follow-up in Period B (Extension Period). In Period B (Extension Period), the participants will receive study intervention at 3 dosing visits (Week 24, Week 32, and Week 40). Participants who received TOUR006 during Period A (Primary Efficacy Period) will receive placebo. Participants who received placebo during Period A (Primary Efficacy Period) will receive TOUR006 50 mg q8w. During Period B, this dose may be decreased by the sponsor to 20 mg q8w based upon any emerging safety insights (such as from a periodic safety review by the Data Safety Monitoring Board [DSMB]) or based upon the results of the primary analysis from Period A.

Follow-up visits will be completed after the last dosing visit until the last visit at Week 72 for the full study. Masking of the participant and the investigator will remain in place throughout the duration of the study.

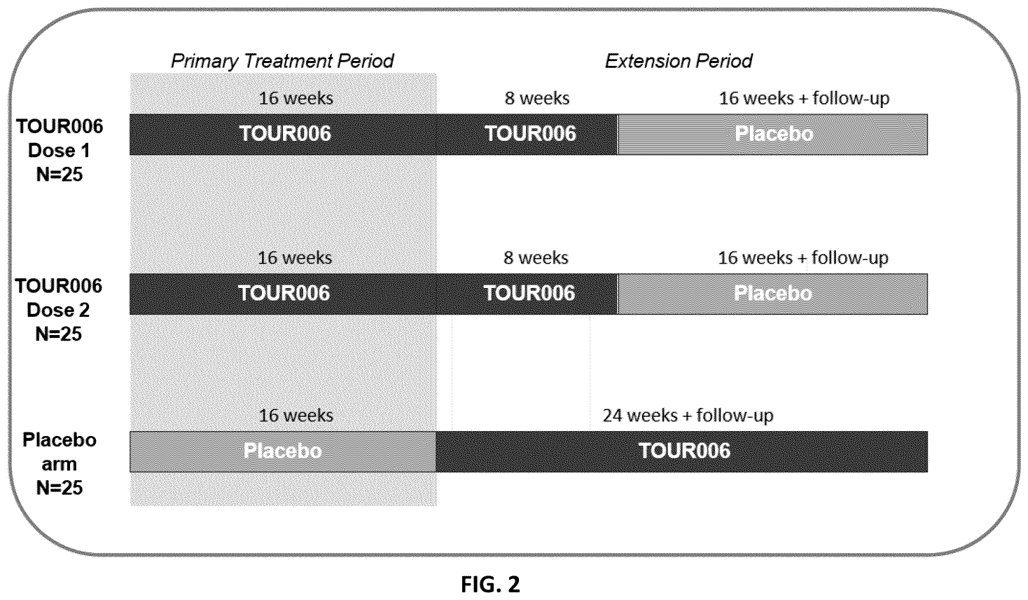

Other study designs might be considered. As shown in FIG. 2, the study will consist of a 16-week primary treatment period (Period A) that is placebo-controlled and an extension period (Period B) in which all patients receive TOUR006. The primary treatment period might be increased to 24 weeks. For Period A, approximately 81 eligible patients (27 per treatment arm) will be randomized in a 1:1:1 ratio to receive SC treatment with TOUR006 (dosing regimen A), TOUR006 (dosing regimen B) or placebo. The patient sample sizes might be adjusted based upon ongoing power calculations. Dosing regimen A will be 50 mg loading dose×1, followed 4 weeks later by 20 mg every 4 weeks for a total of 16 weeks primary treatment. Dosing regimen B will be 20 mg loading dose ×1, followed 4 weeks later by 10 mg every 4 weeks for a total of 16 weeks primary treatment.

After completion of Period A, patients will be offered the option of continuing their study participation by entering Period B. All patients in Period B will be treated with TOUR006 every 4 weeks (up to a maximum cumulative exposure of 24 weeks across Period A and Period B). Patients who have received TOUR006 in Period A will continue receiving the same dose level of TOUR006 with which they have previously been treated. Patients who have received placebo in Period B will be randomized to receive one of the two different dose levels of TOUR006. The double-masking will be maintained for both what a patient have received in Period A as well as what they receive in Period B. After the last treatment visit (Week 36), patients will undergo additional visits for further follow-up until Week 72.

Other dosing regimens such as 20, 25, 30, or 50 mg every 12 week for a total of 24 weeks treatment may be contemplated. Moreover, additional loading dosing regimens might be considered. For example, a loading dosing regimen will be 50 mg loading dose ×1, followed 4 weeks later by 20 mg, 25 mg or 30 mg every 12 weeks; 30 mg loading dose ×1, followed 4 weeks later by 20 mg, 25 mg or 30 mg every 12 weeks; or 25 mg loading dose ×1, followed 4 weeks later by 10 mg, 20 mg or 25 mg every 12 weeks.

Approximately 25 sites in Canada, Jordan, Mexico, United States will participate.

Efficacy assessments will include, but not limited to, proptosis response, CAS, Gorman grading of diplopia, GO-QoL, and autoantibody measurement. Details of these clinically relevant parameters are described in U.S. Pat. Nos. 11,208,489, 11,208,490, Stan et al., Thyroid. 2022 February; 32(2): 170-176, and Bartalena and Wiersinga, Eur Thyroid J 2020; 9 (suppl 1): 3-16, the contents of each of which are hereby expressly incorporated by reference in their entirety for any purpose.

Efficacy assessments will be performed for both eyes at each assessment time point. The most severely affected eye (based upon CAS and proptosis) at baseline will be selected as the “study eye” and will remain so for the duration of the study. The other eye will be designated as the “fellow eye”, which is less severely affected, for the duration of the study. If there is a discrepancy between the CAS and proptosis in the two eyes, the eye with the worse proptosis will be selected as the study eye. If both eyes are equally affected, then the investigator will choose the study eye. Proptosis may be assessed using the same exophthalmometer by the same person per visit. Inflammation may be assessed using the 7-point CAS, which scores the presence of each of the following signs: retrobulbar eye pain, pain on eye movement, eyelid erythema, eyelid swelling, conjunctival redness, chemosis, inflammation of the caruncle or plica. A CAS ≤1 is indicative of disease inactivation. Changes in diplopia grade may be assessed using the Gorman subjective diplopia score (range 0-3). The presence or level of autoantibodies including thyroid stimulating immunoglobulin (TSI) and an anti-thyroid stimulating hormone receptor (TSHR) antibodies may be quantified using methods well known in the art, such as Roche Elecsys anti-TSHR assay (ROC-TBII), Quidel Thyretain™ TSI Reporter BioAssay Kit (QUI-TSI), and Otsuka aequorin TSAb assay (OTS-TSI). The normal range for TSI is typically considered to be less than 1.3 or 1.5 international units per liter (IU/L) depending on the laboratory reference range used. However, the normal range may vary slightly depending on the specific laboratory that performs the test. One of skill in the art, for example an ophthalmologist, surgeon or other clinician skilled in the knowledge and treatment of eye disorders would know what is considered as a normal range of TSI levels.

The study protocol mandates physical examinations to be conducted at baseline (Day 1), Week 8, Week 16, Week 24, Week 32, Week 40, Week 48, Week 64, and Week 72. Additionally, ophthalmic examinations, vital signs checks, and clinical safety laboratory tests will be performed during each visit. Electrocardiograms will be taken at baseline (Day 1), Week 12, Week 24, Week 36, Week 48, and Week 72. Participants will be required to have a post dose safety contact within 24 hours of each injection to ensure safety monitoring.

All adverse events/serious adverse events (AE/SAE) will be recorded from the signing of the informed consent form until last follow-up visit. Any concomitant therapies and interventions will also be recorded.

Adverse events of special interest (AESI) include the following:
Clinically significant infections:
   Infections that meet any SAE criteria
   Confirmed opportunistic infections
   Infections requiring prolonged medications (>14 days)
   Infections requiring any parenteral treatment
Transaminase elevations >3×ULN
Neutrophil count <1000/mm3
Platelet count <50,000/mm3
Thromboembolic events

Each participant will undergo regular safety assessments by the investigator. Ongoing safety evaluation will be conducted by the investigator and sponsor. In addition, an external DSMB and an internal safety management committee (SMC) will meet regularly to monitor the safety of participants and make recommendations regarding the continuation or changes to the study (such as pausing enrollment across the study or for a particular dose level, pausing dosing across the study or for a particular dose level, stopping a dose level, requesting additional safety analyses, or adding new or more frequent safety assessments to the safety monitoring for each study participant). Periodic safety reviews will include a review of treatment-emergent AE, SAE, and AESI.

### Study Endpoints

The primary endpoint is the percentage of participants achieving a proptosis response, which is defined as a reduction of ≥2 mm from baseline in the study eye, without increased of ≥2 mm proptosis in the fellow eye and without the need for rescue therapy/intervention, in the TOUR006 treatment groups compared with the placebo group at Week 20. The percentage of the proptosis reduction is at least 40%, 50%, 60%, 70%, 75%, or 80%.

The key secondary endpoint is the percentage of participants attaining a complete or near-complete CAS response, which is defined as a CAS of ≤1 in the study eye, without a ≥2 point increase in CAS from baseline in the fellow eye, and without the need for rescue therapy/intervention, in the TOUR006 treatment groups compared with the placebo group at Week 20. The percentage of the CAS reduction is at least 50%, 60%, 70%, 75%, 80%, 85% or 90%.

Other secondary endpoints, including comparison of TOUR006 treatment groups with the placebo group for various time points through Week 24, are as follows:
The percentage of participants achieving a proptosis response in the TOUR006 treatment groups at Week 8, Week 16, Week 24, Week 32, Week 40, Week 44, Week 48, Week 56, Week 64, and Week 72.
Mean change from baseline in proptosis in the study eye at Week 8, Week 16, Week 20, Week 24, Week 32, Week 40, Week 44, Week 48, Week 56, Week 64, and Week 72.
The percentage of participants attaining a complete or near-complete CAS response at Week 8, Week 16, Week 24, Week 32, Week 40, Week 44, Week 48, Week 56, Week 64, and Week 72.
Mean change from baseline in CAS in the study eye at Week 8, Week 16, Week 20, Week 24, Week 32, Week 40, Week 44, Week 48, Week 56, Week 64, and Week 72.
The percentage of participants attaining ≥1 grade decrease in diplopia at Week 8, Week 16, Week 20, Week 24, Week 32, Week 40, Week 44, Week 48, Week 56, Week 64, and Week 72.
The percentage of participants attaining resolution in diplopia at Week 8, Week 16, Week 20, Week 24, Week 32, Week 40, Week 44, Week 48, Week 56, Week 64, and Week 72.
The percentage of participants with inconstant diplopia at baseline attaining resolution of inconstant diplopia at Week 8, Week 16, Week 20, Week 24, Week 32, Week 40, Week 44, Week 48, Week 56, Week 64, and Week 72.
The percentage of participants with constant diplopia at baseline attaining resolution of constant diplopia at Week 8, Week 16, Week 20, Week 24, Week 32, Week 40, Week 44, Week 48, Week 56, Week 64, and Week 72.
Mean Graves' orbitopathy-quality of life (GO-QoL) score at Week 20, Week 44, and Week 72.
Incidence of treatment-emergent adverse events (AEs) by severity and serious AEs (SAEs) through Week 72.

The pharmacokinetic/pharmacodynamic endpoints are as follows:
The effects of TOUR006 over time on absolute serum concentrations of TOUR006.
Pharmacokinetic parameters of TOUR006.
The effects of TOUR006 over time on absolute concentrations for serum high-sensitivity C-reactive protein (hs-CRP).
The effects of TOUR006 over time on absolute concentrations for serum thyroid stimulating immunoglobulin (TSI).
The effects of TOUR006 over time on absolute concentrations for serum thyroid hormone receptor antibody (TRAb).
The effects of TOUR006 over time on absolute concentrations for serum interleukin (IL)-6.
Mean change from baseline in serum concentration of TOUR006.
Mean change from baseline in serum hs-CRP.
Mean change from baseline in serum TSI.
Mean change from baseline in serum TRAb.
Mean change from baseline in serum IL-6.
Percentage of participants with anti-drug antibody (ADA).

The secondary end point further includes a mean improvement in GO-QoL from baseline by at least 6, 8, 10, 15, or 20 points.

The treatment result may be achieved within 72 weeks, 64 weeks, 56 weeks, 48 weeks, 44 weeks, 40 weeks, 32 weeks, 20 weeks, 16 weeks, 12 weeks, 8 weeks, or 4 weeks. The treatment result may also be achieved during a long-term treatment (e.g. more than 24 weeks, more than 48 weeks, more than 72 weeks, or more than 96 weeks after treatment initiation).

The efficacy effect of the treatment is sustained for at least 4 weeks, 8 weeks, 12 weeks, 16 weeks, 24 weeks, 36 weeks, 48 weeks, or 72 weeks after the last dose has been administered. The probability of relapse (i.e., loss of proptosis response or CAS response or diplopia response) is less than 40%, 30%, 20%, 15%, 10%, or 5%.

It is expected that patients started on TOUR006 early will have a better response and/or longer-term outcomes than when started later. The efficacy outcomes of patients who had received TOUR006 in the primary treatment period (i.e., Part A of the study) would be compared to those of patients who had received placebo in the primary treatment period and then later switched to TOUR006 in the Extension Period. It is expected that patients who had started on TOUR006 from the very beginning have better efficacy outcomes (such as at Weeks 32, 36, 40, 48, 72 or 96) than the placebo patients who started TOUR006 only after a 24 weeks delay.

Additional efficacy end points include Thyroid Associated Orbitopathy Scale (TAOS); Bahn Gorman Diplopia Score; TED-QOL; NO SPECS Classification (No physical signs or symptoms, Only signs, Soft tissue involvement, Proptosis, Extraocular muscle signs, Corneal involvement, and Sight loss); European Group on Graves Orbitopathy severity scale (EUGOGO); VISA Classification (vision, inflammation, strabismus, and appearance); 10-point version of CAS; and/or Total Motility Score (TMS).

The biomarker endpoints include a mean or median reduction of Thyroid stimulating immunoglobulin (TSI) serum (or other blood sample) concentrations from baseline by at least 30%, 40%, 50%, 60%, 70%, 80%, or 90%; a mean or median reduction of thyroid-stimulating hormone receptor (TSHR) antibody serum (or other blood sample) concentrations from baseline by at least 30%, 40%, 50%, 60%, 70%, 80%, or 90%; and/or a mean or median reduction of high-sensitivity CRP (hsCRP) or CRP serum (or other blood sample) concentrations from baseline by 50%, 60%, 70%, 80%, or 90%. The probability of TSI suppression to normal range is at least 30%, 40%, 50%, 60%, 70%, 80%, or 90%. The probability of TSHR antibody suppression to normal range is at least 30%, 40%, 50%, 60%, 70%, 80%, or 90%. The probability of hsCRP or CRP suppression to normal range is at least 50%, 60%, 70%, 80%, or 90%. The probability of reduction from baseline in hsCRP or CRP by at least 90% is at least 50%, 60%, 70%, 80%, or 90%.

Additional biomarkers may include Red blood cell distribution width (RDW), IL-6, FGF-23, ESR, fibrinogen, SAA, IL-4, IL-10, IL-12, IL-13, IL-17, IL-23, IL-1β, sIL-1RA, IFN-γ, TGFβ, and TNFα. For each of these biomarkers, a positive effect of TOUR006 could be to induce a decrease in the level.

Adverse effects/toxicities, such as hearing impairment or loss, hyperglycemia, new onset or worsening of diabetes mellitus, hair loss/alopecia, infusion reactions or other hypersensitivity reactions, would be monitored. Tolerability issues, such as muscle spasm, muscle pain, nausea, diarrhea, fatigue, dysgeusia (taste impairment), dry skin, or injection site reactions, would also be monitored. It is expected that TOUR006 provides therapeutic effect in patients with TED while minimizing adverse effects/toxicities. It is expected that TOUR006 will demonstrate a lower rate of adverse effects/toxicities compared to placebo, or that the adverse effects/toxicities that do occur will be substantially lower in severity or frequency than those seen with placebo. It is also expected that TOUR006 has good tolerability in patients with TED. This means that it will be well-tolerated by patients, and there will be no significant issues with patient compliance or discontinuation of therapy.

### Study Population and Sample Size

The study will enroll male and female participants, between the ages of 18 to 75 years old (inclusive) who have with moderate to severe TED. The screening process will involve approximately 93 participants, with the goal of ultimately enrolling 81 participants who will be assigned to investigational intervention. Each treatment group will consist of 27 participants.

### Eligibility Criteria

Participants are eligible to be included in the study only if all the following criteria apply:

### Age

1. Participant must be 18 to 75 years of age inclusive, at the time of signing the informed consent.

### Type of Participant and Disease Characteristics

2. Clinical diagnosis of Graves' disease associated with active TED.
3. Moderate to severe active TED (not sight-threatening but has an appreciable impact on daily life) as assessed by the investigator, usually associated with one or more of the following: lid retraction >2 mm, moderate or severe soft tissue involvement, exophthalmos (proptosis), and/or inconstant or constant diplopia.
4. Onset of active TED symptoms (as determined by medical history) within approximately 12 months before screening and with both of the following criteria also met:
   a. CAS ≥4 (on the 7-item scale) for the study eye at screening and reconfirmed at baseline.
   b. Exophthalmos (proptosis) ≥3 mm above the normal range (based upon race and gender) for the study eye at screening and reconfirmed at baseline.
5. Presence of TSI >130% of the normal range.
6. Participants must be euthyroid with the baseline disease under control or have mild hypo- or hyperthyroidism (defined as FT4 and FT3 levels <50% above or below the normal limits and not associated with any clinically significant and unstable symptoms or complications other than TED) at screening.
7. The eye disease is not anticipated to lead to the emergence of sight-threatening complications, significant and acute deterioration in vision, or require surgical intervention within the treatment Period A (Primary Efficacy Period). Examples of such significant and acute visual deterioration may include: decrease in best corrected visual acuity by 2 or more lines assessed with the Snellen chart over the course of the study, new visual field defect, or color defect secondary to optic nerve involvement.

### Weight

8. Body Mass Index ≤35.0 kg/m²

### Sex and Contraceptive/Barrier Requirements

9. Contraceptive use by females and males should be consistent with local regulations regarding the methods of contraception for those participating in clinical studies.

### Male Participants:

Male participants must be surgically sterile or, if sexually active with a female partner of childbearing potential must also agree to use adequate contraception for the duration of the study, and 4 months after completion of study intervention administration.

### Female Participants:

Females of childbearing potential (including those with an onset of menopause <2 years before screening, non-therapy-induced amenorrhea for <12 months before screening, or not surgically sterile [absence of ovaries and/or uterus]) must have a negative serum pregnancy test at screening, negative urine pregnancy test at all protocol-specified timepoints, and agree to use at least 1 acceptable method of contraception throughout the trial and for 32 weeks after the last dose of study intervention. Female participants who are expected to have reached reproductive maturity by the end of the trial must agree to adhere to trial-specific contraception requirements.

### Informed Consent

10. Capable of giving signed informed consent, which includes compliance with the requirements and restrictions listed in the informed consent form and in the protocol.

### Other Inclusion Criteria

11. Participant is willing and able to comply with the prescribed treatment protocol and evaluations for the duration of the study.

Participants may not enter the study if any of the following exclusion criteria is present:

### Medical Conditions

1. Decreased best corrected visual acuity due to optic neuropathy as defined by a decrease in vision of 2 lines on the Snellen chart, new visual field defect, or color defect secondary to optic nerve involvement within the last 6 months before screening.
2. Monocular vision: defined as best corrected visual acuity less than 20/400 in the fellow eye, or a serious ocular condition in the fellow eye that could lead to worsening of vision.
3. Corneal decompensation unresponsive to medical management.
4. Previous orbital irradiation or surgery for TED.
5. Identified pre-existing ophthalmic disease that, in the judgment of the investigator, would preclude study participation or confound interpretation of study results.
6. CAS <4 at baseline or a decrease in CAS of ≥2 points between screening and baseline in the study eye.
7. Exophthalmos (proptosis)<3 mm above the normal range (based upon race and gender) for the most severely affected eye at screening and reconfirmed at baseline, or decrease in proptosis of ≥2 mm between screening and baseline in the study eye.
8. Immunodeficiency (genetic or acquired, such as acquired immunodeficiency syndrome, common variable immunodeficiency, etc).
9. Serious infection (an infection requiring hospitalization and/or intravenous (IV) antibiotic, IV antifungal, or IV antiviral treatment and/or having a clinical presentation that is viewed by the investigator as consistent with a serious infection) within the past 12 months before screening or more than 1 such episode over the past 36 months before screening.
10. Presence of a transplanted organ.
11. Any tuberculosis (TB) history or current evidence of latent or active TB infection, evidence of active TB (or other active infection) by chest radiography, or residing with or frequent close contact with individual(s) with active TB.
12. Active pulmonary infection.
13. Any history of or current serious opportunistic infection (not including localized thrush due to corticosteroid therapy).
14. Any history of or current thromboembolic event, clinically significant laboratory finding of hypercoagulability, or family history of hypercoagulability.
15. Any history of or current documented atrial fibrillation episode, regardless of if paroxysmal or not and regardless of if symptomatic or not.
16. Clinically significant bleeding diathesis or on an anticoagulant therapy.
17. Biopsy-proven or clinically suspected inflammatory bowel disease (e.g., diarrhea with or without blood or rectal bleeding associated with abdominal pain or cramping/colic, urgency, tenesmus, or incontinence for more than 4 weeks without a confirmed alternative diagnosis OR endoscopic or radiologic evidence of enteritis/colitis without a confirmed alternative diagnosis).
18. History of gastrointestinal (GI) perforation or abscess.
19. History of or clinically suspected systemic lupus erythematosus.
20. Pre-existing demyelinating disorder such as multiple sclerosis
21. History of new onset seizures, unexplained sensory, motor, or cognitive, behavioral or neurological deficits within the last 12 months before screening.
22. Any other significant concurrent medical conditions at the time of screening or baseline visit, including, but not limited to, the following:
   a. Any major illness/condition or evidence of an unstable clinical condition (e.g., renal, hepatic, hematologic, GI, endocrine, cardiac, pulmonary, immunologic, infection, rheumatologic, etc.) that, in the investigator's judgment, will substantially increase the risk to the subject, or confound the interpretation of safety assessments, if they were to participate in the study;
   b. Cancer or history of cancer or lymphoproliferative disease within the previous 5 years (other than resected cutaneous basal cell or squamous cell carcinoma that has been treated with no evidence of recurrence) and not under current treatment for cancer or lymphoproliferative disease;
   c. Class II, III or IV congestive heart failure as defined by the New York Heart Association;
   d. Any history of acute coronary syndrome (myocardial infarction, unstable angina pectoris, or need for urgent coronary revascularization) or cerebrovascular event within 5 years before screening;
   e. Moderate or severe COVID-19 infection within past 90 days before screening, or mild COVID-19 infection within past 30 days before screening;
   f. Persistent symptoms or sequelae following COVID-19 infection, such as Long COVID or cardiovascular complications of COVID-19 infection;
   g. Any history of mpox infection;
   h. Any vaccination within 30 days before screening.
23. Pregnant or lactating.
24. Any major surgery (e.g., requiring general anesthesia or would lead to significant recovery time that would impede participation in the clinical trial or confound the interpretation of the safety results) scheduled or expected during the study.
25. Any ocular surgery (regardless of need for general anesthesia or significant recovery time) scheduled or expected during the study, such as cataract, laser peripheral iridotomy, refractive, or retinal detachment surgery.
26. Any history of or current serious psychiatric disorder or alcohol/drug abuse that in the opinion of the investigator may impact the assessment of safety, efficacy, or protocol adherence.
27. Any other condition that, in the opinion of the investigator, would impair the ability of the participant to comply with the study procedures or impair the ability to interpret data from the participant's participation in the study.

### Prior/Concomitant Therapy

28. History of systemic (e.g., oral or IV) steroid use with a cumulative dose equivalent to ≥1 g of methylprednisolone for the treatment of TED; previous oral steroid use with a cumulative dose of <1 g methylprednisolone (or equivalent dosage for other systemic glucocorticoid) for the treatment of TED is allowed if the corticosteroid was discontinued at least 6 weeks before screening.
29. Any systemic (e.g., oral or IV) corticosteroid use for conditions other than TED within 3 months before screening. (Topical steroids for dermatological conditions, however, are allowed. Inhaled steroids are also permitted as long as no systemic effects are observed or anticipated per investigator's opinion. Steroid-containing eye drops are not permitted).
30. Eye drops with anti-inflammatory activity (e.g., steroid eye drops or cyclosporine eye drops) are not permitted. The use of other topical eye treatments as supportive care (e.g., artificial tears or non-steroidal eye drops, gels or ointments) in accordance with standard of care are permitted.
31. Selenium and biotin must be discontinued 3 weeks before screening and must not be restarted during the trial; however, taking a multivitamin that includes selenium and/or biotin is allowed.
32. Any previous treatment with teprotumumab.
33. Any previous treatment with an immunomodulatory or immunosuppressive agent within 5 half-lives of the drug or 3 months (whichever is longer) before screening.
34. Use of an investigational agent for any condition within 5 half-lives of the drug or 3 months (whichever is longer) before screening.
35. Any prior exposure to a monoclonal antibody, Fc-bearing protein, or other protein therapy.
36. Any anticipated use over the duration of the study of a prohibited concomitant therapy (e.g., systemic corticosteroids, a biologic other than study intervention, or immunosuppressant such as methotrexate, azathioprine, 6-MP, mycophenolate mofetil, etc).
37. Any planned live (attenuated) vaccination during the course of this study.

### Prior/Concurrent Clinical Study Experience

38. Known hypersensitivity to any of the components of TOUR006 or prior hypersensitivity reactions to monoclonal antibody or other Fc-bearing protein.
39. Known prior exposure to this investigational agent.

### Diagnostic Assessments

40. Abnormality in any of the following testing assessments at screening as outlined below:
   a. Positive HIV test
   b. Positive HBV test that is consistent with current or prior infection (note: positive Hepatitis B surface antibody test consistent with vaccination but no infection is not an exclusion criterion)
   c. Positive HCV test
   d. Positive TB test or chest x-ray abnormality suspicious for TB or other infection
   e. ALT or AST levels ≥1.5× ULN
   f. Total bilirubin level ≥1.5 times the ULN
   g. Hemoglobin level <10.0 g/dL
   h. Platelet count ≤100×109/L (100,000 cells/mm³) or ≥1000 ×109/L (1,000,000 cells/mm³)
   i. White blood cell count ≤3.5×109/L (3500 cells/mm³)
   j. Absolute neutrophil count (ANC) <2000 cells/mm³
   k. Serum creatinine level ≥177 μmol/L (2 mg/dL)
   l. Uncontrolled diabetes, defined as a hemoglobin A1c >8% at screening
   m. Uncontrolled hyperlipidemia defined as fasting LDL-C of >130 mg/dl or non-fasting of LDL-C >190 mg/dl at screening
   n. Uncontrolled hypertension, defined as a systolic value ≥140 mm Hg and/or diastolic value ≥90 mm Hg at screening, confirmed on 2 measures >30 minutes apart.

### Statistical Methods

Approximately 81 participants will be randomly assigned to investigational intervention. The sample size calculation is based on the primary efficacy estimand.

It is assumed that the proportion of participants achieving proptosis response in the fellow eye and without need for rescue therapy/intervention at Week 20 is 70% in the 50 mg TOUR006 treatment group, and is 20% in the placebo group. A total of 46 evaluable participants from both 50 mg TOUR006 and placebo (considering 1:1 randomization ratio) will power with 90% the pairwise comparison to detect a treatment difference on TOUR006 and placebo using Fisher's exact test, at a 2-sided alpha level of 5%. Considering power loss by study terminations, the number of randomized participants per group is defined as 27.

There is no interim analysis planned. However, the data of Period A (Primary Efficacy Period) and Period B (Extension Period) will be analyzed at different timepoints of the final analysis of Period A (Primary Efficacy Period), Period B (Extension Period) will be ongoing.

The main estimands for the primary efficacy endpoint (percentage of participants attaining proptosis response at Week 20) and the key secondary efficacy endpoint (percentage of participants attaining complete or near-complete CAS response at Week 20) comparing the respective TOUR006 treatment group with placebo, will be tested following the hierarchical approach with a type-1 error alpha of 5% for the two-sided test on each test level. The sequence of hypotheses will be as follows:
the comparison of the TOUR006 50 mg treatment group versus placebo for the proptosis response,
the comparison of TOUR006 50 mg treatment group versus placebo for the CAS response,
the comparison of TOUR006 20 mg treatment group versus placebo for the proptosis response, and
the comparison of TOUR006 20 mg treatment group versus placebo for the CAS response.

If the TOUR006 50 mg treatment group is discontinued for safety reasons by the DSMB or sponsor and prior to the timing of the primary analysis for efficacy, then the aforementioned sequence of hypotheses will start with the comparison of TOUR006 20 mg treatment group versus placebo for the proptosis response, without any alpha adjustment.

In the primary analysis, the treatment groups will be compared on the intention-to-treat after imputation of missing values using Mantel-Haenszel test for common risk difference considering the randomization stratification factor baseline proptosis (<23 mm versus ≥23 mm).

Proptosis measurements, CAS and diplopia measurements will be summarized at all scheduled visits with actual values and change from baseline by treatment group and study period.

GO-QoL total scores and subscale scores will be summarized at all scheduled visits with actual values and change from baseline by treatment group and period.

Statistical comparison between each TOUR006 dose group and placebo will be performed using the Mixed Model Repeated Measures for the change from baseline assessments and using the Generalized Linear Mixed Model for binary data.

Safety and tolerability parameters will be listed by treatment and participant and displayed in summary tables using descriptive statistics. Safety analysis will be summarized descriptively by treatment arms and study period.

Clinical laboratory data, vital signs, and 12-lead electrocardiogram (ECG) parameters will be summarized using descriptive statistics, including mean values and mean change from baseline values for each scheduled visit by treatment group and study period. Additionally, clinical laboratory data will be presented with the number and percentage of participants with values below, within, or above the normal range at each scheduled visit. For laboratory data and the ECG overall interpretation, frequency tables will be provided with the number and percentage of participants with normal/abnormal/clinically significant abnormal values compared to baseline.

Ophthalmic and physical examination results will be listed.

Summary tables by treatment group will be provided for concomitant treatment and intervention by study period.

The TOUR006 concentration will be summarized by scheduled assessment times and dose level by study period.

The PD parameters hs-CRP and IL-6 will be summarized for actual values and change from baseline by scheduled assessment times and treatment group by study period.

ADA and available neutralizing antibody results will be summarized by scheduled assessment times and TOUR006 dose level by study period.

### Study Rationale

A study population of generally treatment-naïve participants in the active phase of disease supports the investigation of first-line use. In addition, this approach enriches the population for participants with active inflammation who may be more likely to respond to anti-inflammatory therapy.

The primary efficacy endpoint of proptosis response, along with the secondary endpoints of CAS and diplopia, are all clinically meaningful and relevant to the evaluation of TED treatment. The primary endpoint of proptosis response is objective and validated, with a clinically meaningful response threshold of ≥2 mm decrease from baseline.

The 24-week duration of Period A (Primary Efficacy Period) is appropriate to assess treatment effect and is consistent with prior randomized clinical trials in TED as well as the time course for the onset of effect reported in published clinical observations of tocilizumab (TCZ) (IL-6 pathway inhibitor) in TED. The duration of Period B (Extension Period) is also appropriate to evaluate long-term outcomes (72 weeks from start of therapy for participants randomized to TOUR006 in Period A and 48 weeks from start of therapy for placebo participants reassigned to TOUR006 in Period B).

The inclusion of placebo, along with double-masking, in Period B is to mitigate against the introduction of bias in the assessment of efficacy and safety in Period A. This approach helps ensure that study sites and investigators remain blinded to the treatment received by participants in Period A, which could potentially influence their conduct or assessment of other participants who are still in Period A of the study.

### Rescue Therapy and Intervention

In the event of significant and acute clinical deterioration in a participant's TED during the study, the participant may be treated with rescue therapy or other intervention that is consistent with local standard of care. Examples of local standard of care therapy/intervention that may be permitted include, but not limited to, systemic glucocorticoids, teprotumumab, orbital decompression or other surgical procedure, and orbital irradiation.

Participants may receive rescue therapy/intervention only if at least one of the following criteria for significant and acute clinical deterioration in their TED are met, as assessed by the investigator:
Sight-threatening complication (e.g., optic nerve compression)
Significantly and acutely worsening vision that may otherwise become irreversible, such as: decrease in best corrected visual acuity by 2 or more lines on the Snellen chart from baseline ophthalmologic examination, new visual field defect, or color defect secondary to optic nerve involvement.
Other sight-threatening deterioration that an investigator determines to warrant the use of rescue therapy/intervention

Rescue therapy/intervention should only be administered in cases of significant and acute clinical deterioration in participants. This means that participants who experience a lack of improvement in their TED or mild/gradual worsening of their disease are not eligible for rescue therapy/intervention.

During Period A of the study, if a participant experiences significant and acute clinical deterioration, the investigator must consult with the medical monitor before initiating any rescue therapy/intervention. In Period B, consultation with the medical monitor is recommended but not required. The decision as to whether or not to initiate rescue therapy/intervention will ultimately be made by the investigator.

If a participant in the study requires rescue therapy or intervention, they will be required to discontinue the study intervention. However, they will still be able to continue in the trial, including the second treatment period, unless they choose to withdraw from the study or the investigator determines that it would be unsafe for the participant to continue.

In addition, concomitant therapies or interventional procedures that are medically indicated for any AEs the participant has during the trial are permitted at the discretion of the investigator and supersede any of the restrictions outlined in the clinical trial protocol. Unless the therapy/intervention is being used for significant and acute clinical deterioration in TED, it would not be considered rescue therapy/intervention for the purposes of data collection or analysis.

### Example 2. PK/PD Modeling of TOUR006

The purpose of this study is to inform dosing parameters for the treatment of TED with TOUR006 using pharmacokinetic/pharmacodynamic (PK/PD) based simulations.

C-reactive protein (CRP) is directly downstream of IL-6 signal transduction, and irrelevant if ligand or receptor is blocked. There is tightly linked temporal association between CRP and IL-6 pathway. Thus, serum CRP is a promising pharmacology marker of IL-6 pathway activity. Using CRP as marker to identify what level of IL-6 pathway suppression associated with the dosing regimen would inform PD goal of TOUR006 for the treatment of TED.

Population modeling analyses were conducted using nonlinear mixed effects modeling. The CRP data for the modeling came from a multiple dose study of TOUR006 in rheumatoid arthritis (RA) patients receiving background methotrexate (NCT00838565) and the clinical investigation of tocilizumab in RA patients (Paccaly et al., J Clin Pharmacol. 2021 January; 61(1): 90-104; Xu et al., J. of Clinical Pharma., 2021, 61(5): 714-724.) The assumption of the modeling is that the background inflammatory state of TED is similar to RA. The published observations of CRP levels in TED are consistent with the model's assumptions. For example, the mean and median concentrations of hsCRP in patients with TED are less than 10 mg/L, with many patients having less than 5 mg/L. (Czarnywojtek et al., Arch Immunol Ther Exp (Warsz). 2014; 62(6): 501-509; Zhang et al., Endocrine Connections, 2022; Vol. 11 (11); Perez-Moreiras et al., Am J Ophthalmol 2018; 195:181-190.).

The PK/PD model explored two subpopulations with relatively less severe and more severe inflammation:
Population A: Baseline CRP >2 mg/L to 10 mg/L
Population B: Baseline CRP >10 mg/L.

The PK/PD based simulations were performed for the dosing scenarios given in Table 2.

TABLE 2
Dosing regimen

50 mg LD, followed by 20 mg Q4W starting at 4 weeks
20 mg LD, followed by 10 mg Q4W starting at 4 weeks
50 mg Q8W
20 mg Q8W
50 mg Q12W
30 mg Q12W
20 mg Q12W
50 mg Q12W starting at 4 weeks
50 mg LD, followed by 50 mg Q12W starting at 4 weeks
20 mg LD, followed by 30 mg Q12W starting at 4 weeks
Note:
“LD” refers to loading dose; “Q” herein refers to “every”; and “W” refers to “week”

The simulation results were calculated for 4, 8, 12, 16, 20, and 24 weeks from the first dose.

The CRP suppression goal is at least 90% decrease from baseline (based upon CRP effects observed from Tocilizumab 8 mg/kg IV q4 weeks in RA). The simulations modeled what percentage of patients attained the CRP suppression goal for a given TOUR006 regimen. Any patient with CRP suppression of less than 2 mg/L after 7 days of treatment was considered to have at least 90% suppression. This was done to avoid ceiling effects (<2 mg/L is entering into the normal range).

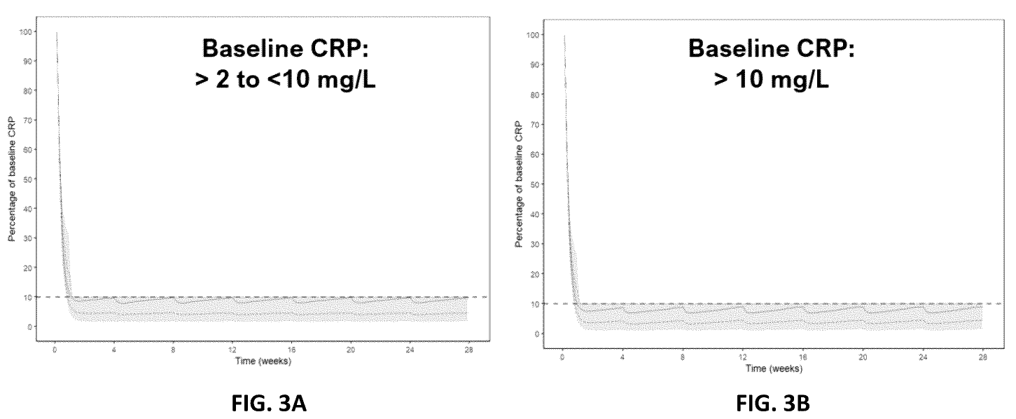

As shown in FIG. 3A and FIG. 3B, the PK/PD modeling predicts that nearly all patients, including population A (FIG. 3A) and population B (FIG. 3B), rapidly attain the CRP suppression goal (≥90% decrease) under the dose regimen of 50 mg LD, followed by 20 mg Q4W starting at 4 weeks.

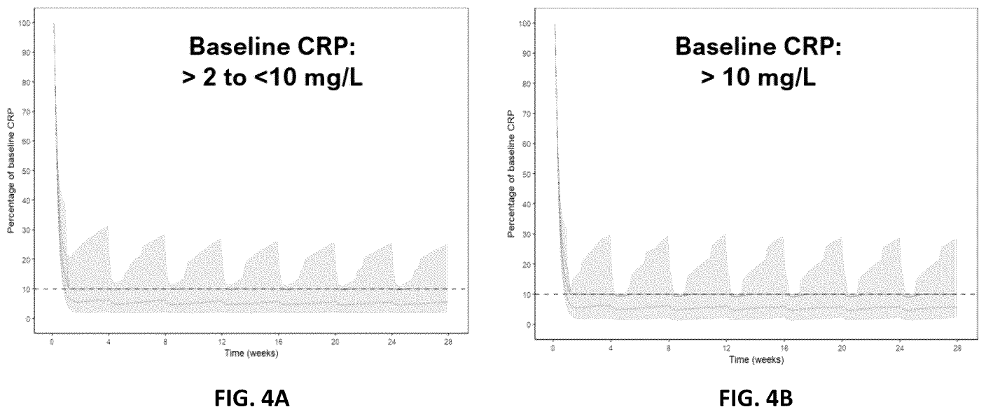

As shown in FIG. 4A and FIG. 4B, the PK/PD modeling predicts that most patients (˜90%), including population A (FIG. 4A) and population B (FIG. 4B), rapidly attain the CRP suppression goal (≥90% decrease) under the dose regimen of 20 mg LD, followed by 10 mg Q4W starting at 4 weeks, although with incomplete population coverage relative to the dose regimen of 50 mg LD, followed by 20 mg Q4W starting at 4 weeks.

Further, the PK/PD modeling predicts rapid and robust CRP suppression for both dose regimens—50 mg LD, followed by 20 mg Q4W starting at 4 weeks, and 20 mg LD, followed by 10 mg Q4W starting at 4 weeks. Table 3 and Table 4 provide the percentage of patients with at least 90% CRP suppression over the treatment period.

TABLE 3
Percent of patients with ≥90% CRP suppression under the dose regimen
of 50 mg LD, followed by 20mg Q4W starting at 4 weeks

| Baseline CRP | Week 4 | Week 8 | Week 12 | Week 16 | Week 20 | Week 24 |
|---|---|---|---|---|---|---|
| >2 to ≤10 mg/L | 94% | 94% | 94% | 94% | 94% | 94% |
| >10 mg/L | 97% | 96% | 96% | 95% | 95% | 95% |

TABLE 4
Percent of patients with ≥90% CRP suppression under the dose regimen
of 20 mg LD, followed by 10 mg Q4W starting at 4 weeks

| Baseline CRP | Week 4 | Week 8 | Week 12 | Week 16 | Week 20 | Week 24 |
|---|---|---|---|---|---|---|
| >2 to ≤10 mg/L | 89% | 90% | 90% | 91% | 91% | 91% |
| >10 mg/L | 89% | 89% | 89% | 89% | 89% | 90% |

Additionally, the PK/PD modeling predicts that less frequent dosing regimens achieve the CRP suppression goal of at least 90% decrease from baseline within the 24 weeks treatment period. Tables 5 to 7 provide the percentage of patients with at least 90% CRP suppression over the treatment period under the less frequent dosing regimens.

TABLE 5
Percent of patients with ≥90% CRP suppression under the dose regimen
of 50 mg Q8W

| Baseline CRP | Week 4 | Week 8 | Week 12 | Week 16 | Week 20 | Week 24 |
|---|---|---|---|---|---|---|
| >2 to ≤10 mg/L | 98% | 95% | 98% | 97% | 98% | 97% |
| >10 mg/L | 95% | 93% | 97% | 95% | 98% | 95% |

TABLE 6
Percent of patients with ≥90% CRP suppression under the dose regimen
of 20 mg Q8W

| Baseline CRP | Week 4 | Week 8 | Week 12 | Week 16 | Week 20 | Week 24 |
|---|---|---|---|---|---|---|
| >2 to ≤10 mg/L | 94% | 88% | 94% | 93% | 95% | 93% |
| >10 mg/L | 91% | 80% | 93% | 85% | 94% | 87% |

TABLE 7
Percent of patients with ≥90% CRP suppression under the dose regimen
of 50 mg Q12W

| Baseline CRP | Week 4 | Week 8 | Week 12 | Week 16 | Week 20 | Week 24 |
|---|---|---|---|---|---|---|
| >2 to ≤10 mg/L | 99% | 99% | 94% | 100% | 98% | 94% |
| >10 mg/L | 99% | 96% | 94% | 99% | 96% | 94% |

The PK/PD modeling predicts effective dosage arms for TED Phase 2b trial. Specifically, a dosing regimen of 50 mg LD, followed by 20 mg Q4W starting at 4 weeks is predicted to result in 94-98% of patients to achieve target CRP suppression in both the moderate inflammation (i.e., baseline CRP 2 to 10 mg/L) and severe inflammation (i.e., baseline CRP of >10 mg/L) populations. A dosing regimen of 20 mg LD, followed by 10 mg Q4W starting at 4 weeks is predicted to result in ˜90% of patients to achieve target CRP suppression in both the moderate inflammation and severe inflammation populations. Both regimens are predicted to achieve rapid (i.e., in 2 weeks or less) suppression of CRP by ≥90% from baseline.

The PK/PD modeling also predicts that less frequent dosing regimens offer opportunity for robust CRP suppression while further reducing drug administration burden for patients. Specifically, the less frequent dosing regimens including 50 mg Q8W and 50 mg Q12W regimens are both predicted to offer CRP suppression effects similar to the 50 mg LD, followed by 20 mg Q4W starting at 4 weeks regimen. The 20 mg Q8W regimen is predicted to offer CRP suppression effects similar to the regimen of 20 mg LD, followed by 10 mg Q4W starting at 4 weeks.

The PK/PD modeling results predict that less frequent or lower dose regimens offer similar CRP suppression effects, particularly if a loading dose is used.

The PK/PD modeling results further suggest a potential improvement in patient experience and health outcomes. The dosing regimens for the phase 2b study (50 mg LD or 20 mg LD) planned for the treatment of TED are multi-fold lower than chronic regimens evaluated previously in the TOUR006 development program in Crohn's disease (CD) (NCT01345318), indicating desirable clinical response outcomes due to the lower doses being used. For example, 50 mg dose is 2-fold less than and 20 mg dose is 5-fold less than the 100 mg dose evaluated in the Crohn's extension study, respectively. In addition, it is predicted that the treatment duration in TED will be finite and limited (e.g., 6 months) and thus further mitigates risk for adverse effects that are dependent upon exposure duration.

The PK/PD Modeling supports using TOUR006 to offer a patient-centric treatment approach in TED through a low drug administration burden. Table 8 provides the dosing regimens of Teprotumumab and TOUR006 for comparison.

TABLE 8
The dosing regimens of Teprotumumab and TOUR006

| | | | Total # of |
|---|---|---|---|
| | | | Treatment |
| | Route of | Treatment | Visits for 6 |
| Treatment Regimen | Administration | Setting | Month Course |
| Teprotumumab q3 | IV | IV infusion | 8 |
| week | | center | |
| TOUR006 q4 week | SC | Home or clinic | 6 |
| TOUR006 q8 week | SC | Home or clinic | 3 |
| TOUR006 q12 week | SC | Home or clinic | 2 |

Overall, the PK/PD Modeling predicts that the planned dosing regimens of TOUR006 offer broad, deep and durable effects, an appropriate safety profile and low drug administration burden which supports a patient-centric treatment experience.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1-34. (canceled)

35. A method of treating thyroid eye disease (TED) comprising administering to a patient in need thereof a therapeutically effective dose of an anti-interleukin-6 (anti-IL-6) antibody or antibody fragment having the variable heavy (VH) CDRs as defined in SEQ ID NOs 2, 3 and 4, and the variable light (VL) CDRs as defined in SEQ ID NOs 8, 9 and 10.

36. The method of claim 35, wherein the anti-IL-6 antibody or antibody fragment comprises a heavy chain polypeptide comprising a polypeptide having at least 95% identity to SEQ ID NO: 1 and a light chain polypeptide comprising a polypeptide having at least 95% identity to SEQ ID NO: 7.

37. The method of claim 35, wherein the anti-IL-6 antibody or antibody fragment comprises a heavy chain polypeptide having the sequence of SEQ ID NO: 1 and a light chain polypeptide having the sequence of SEQ ID NO: 7.

38. The method of claim 35, wherein the anti-IL-6 antibody or antibody fragment is administered in a pharmaceutical composition that comprises said anti-IL6 antibody or antibody fragment and a pharmaceutically acceptable carrier.

39. A method of treating thyroid eye disease (TED) comprising administering to a patient in need thereof a therapeutically effective dose of an anti-interleukin-6 (anti-IL-6) antibody or antibody fragment having the variable heavy (VH) CDRs as defined in SEQ ID NOs 2, 3 and 4, and the variable light (VL) CDRs as defined in SEQ ID NOs 8, 9 and 10, wherein the therapeutically effective dose is administered in a pharmaceutical composition which comprises 85 mg/mL of the anti-IL-6 antibody, 20 mM histidine, 63.2 mg/mL of sucrose, 16.8 mg/mL of mannitol, 0.05 mg/mL of EDTA, and 0.2 mg/mL of polysorbate 80.

40. The method of claim 35, wherein said therapeutically effective dose is between 5 mg to 200 mg.

41. The method of claim 40, wherein said therapeutically effective dose is administered subcutaneously.

42. The method of claim 41, wherein said therapeutically effective dose is administered from every week to every 24 weeks.

43. The method of claim 42, wherein said therapeutically effective dose is administered every 8 weeks.

44. A method of treating thyroid eye disease (TED) comprising administering to a patient in need thereof a therapeutically effective dose of an anti-interleukin-6 (anti-IL-6) antibody or antibody fragment having the variable heavy (VH) CDRs as defined in SEQ ID NOs 2, 3 and 4, and the variable light (VL) CDRs as defined in SEQ ID NOs 8, 9 and 10, wherein the therapeutically effective dose is 20 mg or 50 mg, and wherein the therapeutically effective dose is administered every eight weeks.

45. The method of claim 44, wherein the patient receives at least 3 doses of treatment.

46. The method of claim 45, wherein the therapeutically effective dose is 20 mg.

47. The method of claim 46, wherein the therapeutically effective dose is 50 mg.

48. The method of claim 35, wherein the patient has one or more of Graves' disease associated with active TED, inflammation associated with active TED.

49. The method of claim 35, wherein the patient has at least one or more of the following:
(a) an eye with a Clinical Activity Score (CAS) (7-point scale) of ≥4 before treatment,
(b) an eye with proptosis of ≥3 mm above a normal range before treatment,
(c) thyroid stimulating immunoglobulin (TSI) of >130% above a normal range before treatment,
(d) is euthyroid or has mild hypo- or hyperthyroidism, or
(e) has a body mass index of ≤35.0 kg/m².

50. The method of claim 35, wherein said method of treatment achieves one or more of the following results:
(a) a proptosis reduction of ≥2 mm from baseline in the first eye, without a proptosis increase of ≥2 mm in the second eye;
(b) a Clinical Activity Score (CAS) (7-point scale) of ≤1 in the first eye, without ≥2 point increase in CAS from baseline in the second eye;
(c) a diplopia grade reduction by at least 1 using the Gorman diplopia scale;
(d) improvement from baseline in Graves' ophthalmopathy quality of life (GO-QoL) from baseline by at least 6, 8, 10, 15, or 20 points;
(e) a reduction in the titter of an autoantibody; or
(f) improvement in eyelid retraction.

51. The method of claim 50, wherein the autoantibody comprises TSI and TSHR antibody.

52. The method of claim 50, wherein the probability of the proptosis reduction is at least 40%, 50%, 60%, 70%, 80%, or 90%.

53. The method of claim 50, wherein the probability of the CAS reduction is at least 50%, 60%, 70%, 80%, or 90%.

54. The method of claim 50, wherein the probability of resolution of inconstant diplopia is at least 30%, 40%, 50%, 60%, 70%, 80%, or 90%.

55. The method of claim 50, wherein the probability of the constant diplopia reduction is at least 30%, 40%, 50%, 60%, 70%, 80%, or 90%.

56. The method of claim 50, wherein the treatment result is achieved within 72 weeks, 64 weeks, 56 weeks, 48 weeks, 44 weeks, 40 weeks, 32 weeks, 20 weeks, 16 weeks, 12 weeks, or 8 weeks.

57. The method of claim 50, wherein the treatment result is achieved during a long-term treatment.

58. The method of claim 57, wherein the long-term is more than 72 weeks.

59. A pharmaceutical composition for treating thyroid eye disease (TED) comprising a therapeutically effective dose of an anti-interleukin-6 (anti-IL-6) antibody or antibody fragment having the variable heavy (VH) CDRs as defined in SEQ ID NOs 2, 3 and 4, and the variable light (VL) CDRs as defined in SEQ ID NOs 8, 9 and 10, histidine, sucrose, mannitol, EDTA, and polysorbate 80.

60. The pharmaceutical composition of claim 59, wherein the composition comprises 5 mg to 200 mg of the anti-IL-6 antibody.

61. The pharmaceutical composition of claim 59, wherein the composition comprises 85 mg/mL of the anti-IL-6 antibody.

62. The composition of claim 59, wherein the pharmaceutical composition comprises 85 mg/ml of the anti-IL-6 antibody, 20 mM histidine, 63.2 mg/mL of sucrose, 16.8 mg/mL of mannitol, 0.05 mg/mL of EDTA, and 0.2 mg/mL of polyasorbate 80.

63. The composition of claim 59, wherein the composition is at a pH of 5.8.

64. The method of claim 35, wherein treatment efficacy is sustained for at least 4 weeks, 8 weeks, 12 weeks, 16 weeks, 24 weeks, 36 weeks, 48 weeks, 72 weeks or 96 weeks after the last dose has been administered.
